(19) United States
(12) Patent Application Publication
(10) Pub. No.: US 2005/0266394 A1
(43) Pub. Date: Dec. 1, 2005

(54) Magnetophoretic cell clarification

(75) Inventors: T. Alan Hatton, Sudbury, MA (US); Sonja A. Sharpe, Lawrenceville, NJ (US); Seyda Z. Bucak, Cihangir Instanbul (TR); Simon Kuhn, Otterstadt (DE)

Correspondence Address: FOLEY HOAG, LLP;PATENT GROUP, WORLD TRADE CENTER WEST, 155 SEAPORT BLVD, BOSTON, MA, 02110, US

(73) Assignee: Massachusette Institute of Technology, Cambridge, MA (US)

(21) Appl. No.: 11/021,110

(22) Filed: Dec. 22, 2004

Related U.S. Application Data
(60) Provisional application No. 60/533,033, filed on Dec. 24, 2003.

(51) Int. Cl. C12Q 1/00; C12Q 1/70; C12Q 1/04; C12M 1/34

(52) U.S. Cl. 435/4; 436/526; 435/34; 435/5; 435/287.1

(57) ABSTRACT

One aspect of the present invention relates to magnetophoretic devices for cell clarification. In one embodiment, the magnetophoretic device is a counter-current device, comprising a plurality of pairs of magnets selected from the group consisting of permanent magnets and electromagnets; a chain rotated by a motor; tubing; a feed inlet; and a clarified feed outflow. In another embodiment, the magnetophoretic device is a quadrupole device, comprising four magnets selected from the group consisting of permanent magnets and electromagnets; and a cylindrical column. Another aspect of the invention relates generally to a method of separating non-magnetic particles from a magnetic fluid mixture, comprising the steps of combining a magnetic fluid with a sample comprising non-magnetic particles and non-magnetic fluid to form a mixture; subjecting said mixture to a degrading magnetic field; and isolating a portion of the non-magnetic particles from said mixture.

### RELATED APPLICATIONS

This application claims the benefit of priority to U.S. Provisional Patent Application Ser. No. 60/533,033, filed Dec. 24, 2003; the specification of which is hereby incorporated in its entirety.

### BACKGROUND OF THE INVENTION

The use of fermentation processes to produce valuable biological products is becoming commonplace. The increased use of fermentation processes is due primarily to a greater understanding of how genetically to alter bacteria and other cell cultures to produce a product of interest. Once this product is produced in the fermentation tank, however, it must still be removed from the raw fermentation broth and purified. The first step in downstream recovery of the product typically involves removing the cells from the bulk fermentation fluid, a process called cell clarification.

The most common cell clarification techniques used in industry today are centrifugation and membrane filtration; both technologies are fairly well developed. The choice between the two techniques depends on what cell type needs to be removed (yeast, bacteria, fungi, etc.), the concentration of the cells in the broth, the cost sensitivity of the product, the molecular size of the product, the volume of liquid that must be processed, and the associated regulatory requirements, which are particularly important when the final product is for pharmaceutical or food use.

Centrifugation takes advantage of the density difference between the cells and the raw liquid to force the cells to sediment out of the fluid. Centrifugation can clarify feed flows up to roughly 20,000 L/hr, but has the principle disadvantage of large upfront capital costs, high shear stress on the cells, and the inherent danger of high-speed moving parts. Membrane filtration takes advantage of the size difference between the cells and the product of interest to exclude the cells while allowing the smaller molecules or particles of interest to pass through a membrane. Membrane filtration can clarify feeds with a flux through the membrane of up to 250 μm/s (900 L/hr/m²) for microfiltration, which is the filtration type most often used for cell clarification. Membrane filtration has the advantage of being easy to scale up, but, depending on the extent of filtration needed it may be suboptimal due to large operating costs, low flux through the membrane, clogging and fouling of the membrane, and/or the potential need for multiple membrane stages.

The downstream processing costs for the production of biological products typically represent 80-90% of the total production costs. In contrast, the average downstream processing costs in the chemical industry range from 50-70%.

Industrial Applications of Magnetic Fluids

Magnetic fluids have found commercial use in a variety of industrial applications including sealing, damping, and heat transfer (Raj, K. et al., J. Magn. Magn. Mater. 1995, 149, 174-180). Magnetic fluids are commonly used as rotary shaft seals in hard drives because they provide a means of preventing gas leakage while avoiding rubber parts. In this use, rings of magnetic fluid are held in place around the shaft with external magnets that form a high pressure gas barrier (Rosensweig, R. E. Chem. Eng. Prog. 1989, 85, 53-61). Likewise, a film of magnetic fluid held in place with an external magnet is used in place of an oil film in stepper motors to damp vibrations and oscillations as the motor moves (Raj, K. et al., J. Magn. Magn. Mater. 1995, 149, 174-180). The damping properties of magnetic fluids are also used in loudspeakers (Raj, K. et al., J. Magn. Magn. Mater. 1990, 85, 233-245), where they act as an improved coolant fluid due to their high thermal conductivity and their development of magnetically-driven convection cells in the presence of a magnetic field (Rosensweig, R. E. Ferrohydrodynamics; Dover Publications, Inc.: Mineola, N.Y., 1985). The magnetic fluids used in these industrial applications are usually organic-based (Raj, K. et al., J. Magn. Magn. Mater. 1995, 149, 174-180). A relatively new use of cobalt-based magnetic fluids involves the increase of microwave absorption in the heating of nonpolar systems (Holzwarth, A. et al., Ind. Eng. Chem. Res. 1998, 37, 2701-2706).

Biomedical Applications of Magnetic Fluids

Aqueous magnetic fluids have the potential to be used in a range of biomedical applications in which the nanoparticles generally require a coating that provides colloidal stability in the body and is biocompatible. Magnetic fluids with biocompatible stabilizing polymers have been developed as magnetic resonance imaging (MRI) contrast agents that have improved imaging properties in the body compared to conventional ferric salt solutions (Kawaguchi, T. et al., J. Mater. Sci.—Mater. Med. 2002, 13, 113-117; Douglas, T. et al., In Hybrid Organic-Inorganic Composites; Mark, J. E., Lee, C. Y., Bianconi, P. A., Eds.; ACS-Oxford University Press: New York, 1995; Vol. 585, pp. 19-28). Magnetic fluids have also been used in drug delivery applications, which requires the absorption or covalent attachment of drugs to the nanoparticles (Lubbe, A. S. et al., J. Magn. Magn. Mater. 1999, 194, 149-155; Suzuki, M. et al., Biotechnol. Appl. Biochem. 1995, 21, 335-345). Anti-cancer drugs absorbed on the stabilizing layer of magnetite nanoparticles have been directed in vivo to a tumor by applying an external magnetic field to concentrate the magnetic fluid in the affected area (Lubbe, A. S. et al., J. Magn. Magn. Mater. 1999, 194, 149-155). Furthermore, magnetite particles with attached monoclonal antibodies have been developed that are able simultaneously to deliver the antibody and generate heat by applying an alternating magnetic field to the particles (Suzuki, M. et al., Biotechnol. Appl. Biochem. 1995, 21, 335-345).

Cell and Protein Separation Using Magnetic Particles

Magnetic fluids have been applied to many different biological systems to separate both cells (Safarik, I.; Safarikova, M. J. Chromatogr. B 1999, 722, 33-53) and proteins (Bucak, S. et al., Biotechnol. Prog. 2003, 19, 477-484; Hubbuch, J. J. et al., Biotechnol. Bioeng. 2002, 79, 301-313; Tong, X. D. et al., Biotechnol. Prog. 2001, 17, 134-139; Khng, H. P. et al., Biotechnol. Bioeng. 1998, 60, 419-424; DeCuyper, M. et al., Biotechnol. Bioeng. 1996, 49, 654-658). In most biological separation applications, the magnetic nanoparticles are used as tagging-agents for the biological species of interest, which usually has a negligible magnetic moment.

Cell separation with magnetic particles has been reviewed by Safarik and Safarikova (Safarik, I. and Safarikova, M. J. Chromatogr. B 1999, 722, 33-53). Most techniques for cell separation involve functionalizing the magnetic nanoparticles with ligands that bind reversibly to cells. When added to a fermentation broth, for example, the magnetic particles bind specifically to the target cells, which can then be removed by magnetic separation. This technique typically involves the use of 1-5 μm polymer beads that contain imbedded magnetic nanoparticles, such as the commercial product Dynabeads (Safarik, I. and Safarikova, M. J. Chromatogr. B 1999, 722, 33-53). However, these magnetic particles cannot be considered a true magnetic fluid, because their size is quite large, i.e., comparable to the size of the target cells being separated.

In a few cases, true magnetic fluids have been used for cell separation. For example, a magnetic fluid with functionalized maghemite nanoparticles has been used to separate erythrocyte cells (Halbreich, A. et al., Biochimie 1998, 80, 379-390). Erythrocyte cells are many orders of magnitude larger than the nanoparticles, and therefore, they are covered by many nanoparticles. Erythrocyte cells are also diamagnetic, and hence, display magnetic properties.

Proteins, which are significantly smaller than nanoparticles, can be separated with magnetic fluids on the basis of charge interactions (Bucak, S. et al., Biotechnol. Prog. 2003, 19, 477-484; DeCuyper, M. et al., Biotechnol. Bioeng. 1996, 49, 654-658) or on the specificity of ligands attached to the nanoparticles (Hubbuch, J. J. et al., Biotechnol. Bioeng. 2002, 79, 301-313; Tong, X. D. et al., Biotechnol. Prog. 2001, 17, 134-139; Khng, H. P. et al., Biotechnol. Bioeng. 1998, 60, 419-424). Magnetic fluids based on phospholipid-coated magnetite nanoparticles have also been produced that are capable of protein loadings as high as 1200 mg/cm³ of particles (Bucak, S. et al., Biotechnol. Prog. 2003, 19, 477-484).

These magnetic cell-separation techniques rely on a specific functional moiety present on the magnetic particles. These techniques are specific, therefore, to the separation of one particular cell type, and will only work for that particular cell. The functionalization of the magnetic particles also typically involves the use of antigen/antibody combinations, which limits the types of cells that can be separated to those for which known antigen/antibody combinations exist. Notably, there are no reports that describe the use of magnetic fluids to separate cells, regardless of cell type, from bulk fermentation media.

### SUMMARY OF THE INVENTION

One aspect of the present invention relates to magnetophoretic devices for cell clarification. In one embodiment, the magnetophoretic device is a counter-current device, comprising a plurality of pairs of magnets selected from the group consisting of permanent magnets and electromagnets; a chain rotated by a motor; tubing; a feed inlet; and a clarified feed outflow. In another embodiment, the magnetophoretic device is a quadrupole device, comprising four magnets selected from the group consisting of permanent magnets and electromagnets; and a cylindrical column.

Another aspect of the invention relates generally to a method of separating non-magnetic particles from a magnetic fluid mixture, comprising the steps of combining a magnetic fluid with a sample comprising non-magnetic particles and non-magnetic fluid to form a mixture; subjecting said mixture to a degrading magnetic field; and isolating a portion of the non-magnetic particles from said mixture. In certain embodiments, the magnetic fluid comprises an iron nanoparticle. In further embodiments, the magnetic fluid comprises magnetite nanoparticles. In a further embodiment, the magnetic fluid comprises a magnetite nanoparticle coated with a graft copolymer layer. In certain embodiments, the magnetic fluid comprises a protic or aprotic solvent. In further embodiments, the solvent is water. In certain embodiments, the sample to be separated from a magnetic fluid mixture comprises non-magnetic particles. In further embodiments, the non-magnetic particles are cells (e.g. prokaryotic or eukaryotic cells). The present invention provides a method that can be used with cells regardless of their shape, size, density, or type. In addition, the present invention may be used to clarify samples of any volume. In certain embodiments, the degrading magnetic field is provided by a counter-current device. In certain embodiments, the degrading magnetic field is provided by a quadrupole device.

### BRIEF DESCRIPTION OF THE FIGURES

FIG. 1 is a schematic of the motion of a non-magnetic particle in response to the force exerted on the particle from the magnetization of the surrounding fluid in the presence of a non-uniform magnetic field. In a magnetic fluid, non-magnetic particles behave as if they were diamagnetic.

FIG. 2 depicts a general structure of the magnetic fluid of the present invention, which consists of a magnetic nanoparticle core that is stabilized in water by a polymer layer surrounding the core. In certain embodiments, the diameter of the magnetite core is approximately 8-10 nm while the average diameter of the whole particle including the polymer shell is approximately 32 nm. Accordingly, the thickness of the polymer shell is typically 2-3 times the radius of the core.

FIG. 3 is a schematic diagram of magnetic fluids, which comprise a suspension of magnetite particles in water.

FIG. 4 depicts attachment of carboxyl groups to the surface of a magnetite particle. The carboxyl group forms a chelate bidentate structure with surface iron atoms.

FIG. 5 depicts size distribution of magnetic nanoparticles in magnetic fluid using dynamic light scattering, with (a) number average distribution and (b) volume average distribution. The size distribution corresponds to the size of the entire nanoparticle, both magnetite core and polymer shell. In certain embodiments, the average diameter of the magnetite core is approximately 8-10 nm.

FIG. 6 depicts the synthesis of magnetic particles useful in forming an aqueous magnetic fluid. Specifically, chemical coprecipitation of iron(III) and iron(II) to magnetite with the addition of base, with stabilization of the magnetite provided by the PAA-PEO/PPO graft copolymer.

FIG. 7 is a photograph of a counter-current device showing a close up view of the 36 magnet pairs mounted on the rotating chain. Tubing is shown running between the magnet pairs.

FIG. 8 is a schematic diagram of a counter-current device. A feed mixture of magnetic fluid and non-magnetic particles is transported through PVC tubing into the apparatus by a syringe pump. The direction of fluid flow and magnet movement are indicated.

FIG. 9 is a schematic diagram depicting the direction of magnet movement, fluid flow, and non-magnetic particle movement inside the tubing of a counter-current device. In certain embodiments, non-magnetic particles are cells, which are approximately 60 times larger than the magnetic nanoparticles.

FIG. 10 is a photograph of a counter-current device. Depicted is a complete counter-current system, including tubing and syringe pump for pumping the feed fluid through the device.

FIG. 11 is a graph showing the measured magnetic field profile along the axis of the rotating chain in a counter-current device. The boxes at the bottom of the graph represent the position of the magnet pairs. The peaks of strongest magnetic field occur in the center of the magnet pairs, while the troughs of weakest magnetic field occur in the middle of the space separating the magnet pairs.

FIG. 12 is a schematic of the quadrupole design, showing an overall cylindrical geometry that is radially symmetric.

FIG. 13 is a schematic diagram of a quadrupole device detailing the four permanent magnets and their stainless steel housing box, top view, where 1 indicates the magnets, 2 and 3 indicate the stainless steel plates needed to construct the housing box for the magnets, and N/S indicates the polarity of the magnetic field from each magnet in the finished magnet assembly.

FIG. 14 is a schematic diagram of a quadrupole device detailing the four permanent magnets and their stainless steel housing box, side view.

FIG. 15 is a photograph of a magnet assembly for a quadrupole device.

FIG. 16 is a schematic of a cylindrical column used with a magnet assembly of a quadrupole device, shown at two different side views, each at 90 degrees to one another. The units are in millimeters.

FIG. 17 is a photograph of a cylindrical column with tubing attached.

FIG. 18 is a photograph of a quadrupole system with tubing attached.

FIG. 19 is a graph showing the clarification of 1 wt % polystyrene beads and 1 wt % magnetic fluid as the feed. Clarified Feed represents the amount of the polystyrene collected in the fluid that exited the counter-current device, and PS in Collection Tube represents the amount of polystyrene removed from the feed and collected in the collection tube. The mass of polystyrene beads (2.01 μm diameter) is measured in the feed stream, in the outlet stream (Clarified Outflow), and in the collection reservoir (Collected PS).

FIG. 20 is a graph showing the clarification of 1 wt % cells on a dry-cell basis and 1 wt % magnetic fluid as the feed. Clarified Feed represents the amount of cells collected in the fluid that exited the counter-current device, and Cells in Collection Tube represents the amount of cells removed from the feed and collected in the collection tube. The mass of E. coli cells (˜2 μm diameter) is measured in the feed stream, in the outlet stream (Clarified Outflow), and in the collection reservoir (Collected Cells).

FIG. 21 is a vector plot showing the vector lines for the magnetic flux density (B) produced by the specific orientation and magnetization of the four magnets used in the quadrupole device. The magnets modeled were neodymium-iron-boron 35. The length of the arrows represent the strength of the field, with longer arrows indicating higher field strength. The orientation of the arrowheads represents the north and south polarity of the field. The units are Tesla (T).

FIG. 22 is a graph showing magnetic flux density (B) versus radial distance in a quadrupole column. The graph is a 1-D version of the vector line plot shown in FIG. 21. The thick black line corresponds to the values of the magnetic flux density (B) along the diameter of the column, where 0 cm represents the centerline of the column and 1 cm represents the wall of the column next to the magnets. The B values were taken from the same model of the 4 magnets that was used to obtain the vector line plot shown in FIG. 21. There was no difference in the magnitude of the B values between the diameter tangent to the edges of the magnet and the diameter connecting the centers of the magnets; the magnetic field is symmetric around the centerline axis. The thin black line is a parabolic fit to the values predicted by the model of the 4 magnets. A parabolic equation was used to calculate both the magnetic field strength and the gradient of the magnetic field when a quadrupole design was modeled in Matlab.

FIG. 23 is a graph of quadrupole model results showing the cell weight fraction versus radial distance as the feed moves up the length of the cylinder in the absence of added salt, with 0 cm representing the bottom of the cylinder and the top of the magnets, and 18.2 cm representing the top of the cylinder.

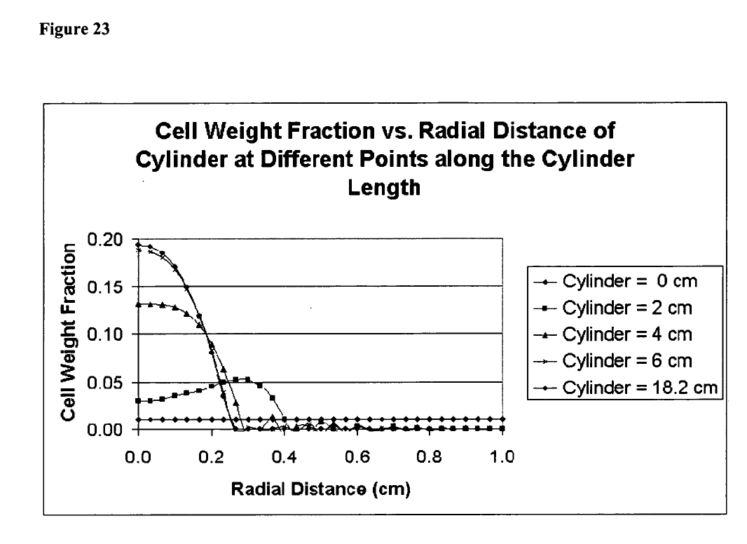

FIG. 24 is a graph of quadrupole model results showing the cell weight fraction versus radial distance as the feed moves up the length of the cylinder in the presence of added salt, with 0 cm representing the bottom of the cylinder and the top of the magnets, and 18.2 cm representing the top of the cylinder.

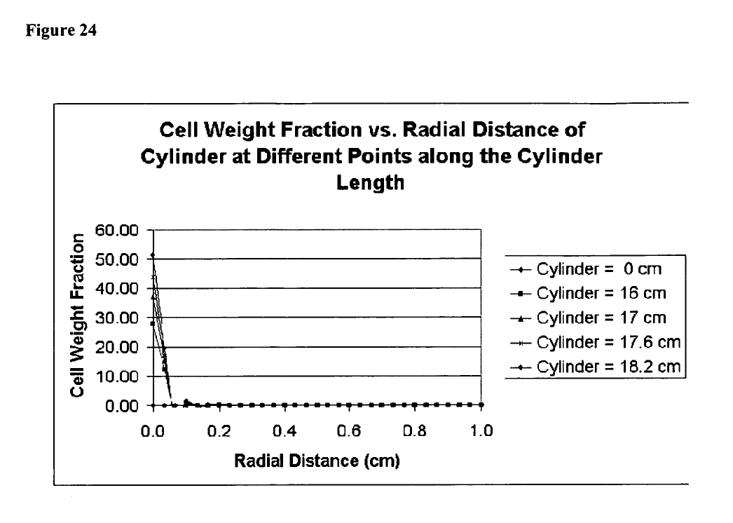

FIG. 25 is a graph of the results of a control experiment using 5 mL of 1 wt % PS as the feed with no magnetic fluid present in the system. Clarified Outflow represents the sum of the PS collected in the two side outlet streams, and Collected PS represents the PS collected from the central outlet stream.

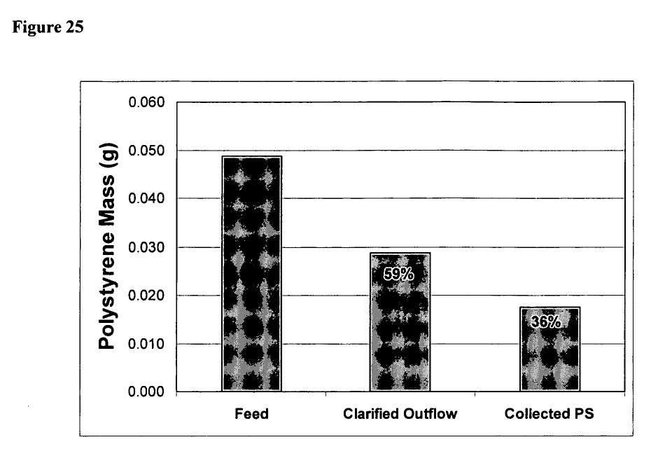

FIG. 26 is a graph of the results of an experiment using 5 mL of 1 wt % PS and 1 wt % MF as the feed with 1 wt % MF present in the system. Clarified Outflow represents the sum of the PS collected in the two side outlet streams, and Collected PS represents the PS collected from the central outlet stream.

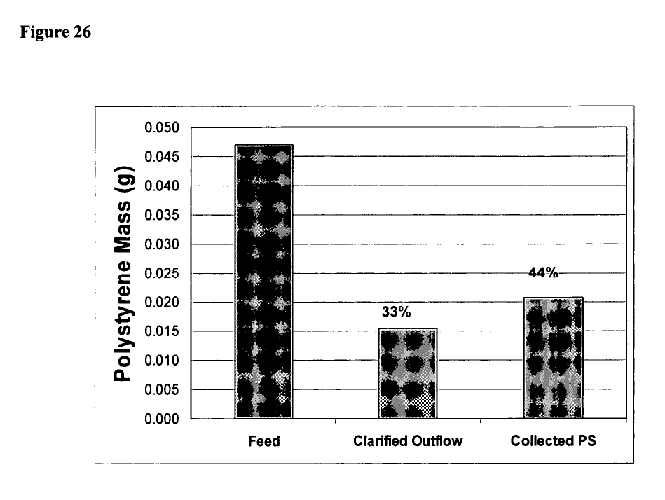

FIG. 27 depicts the dependence of the separation capability of the counter-current device, in terms of the percent of cells removed from the feed fluid, on the dimensionless parameter (Dmd). The dashed line is present to indicate trends in the data.

FIG. 28 depicts the percent of cells removed from the feed in the counter-current device as a function of feed flow rate. The dashed line is present to indicate trends in the data.

FIG. 29 depicts the percent of cells removed from the feed in the counter-current device as a function of magnetic fluid concentration. The dashed line is present to indicate trends in the data.

FIG. 30 depicts the predicted concentration profiles of 2 micron polystyrene beads at different points along the length of the quadrupole column for an initial feed concentration of 1 wt % polystyrene in 1 wt % magnetic fluid with a maximum linear velocity along the centerline of 31.8 cm/hr (50 mL/hr).

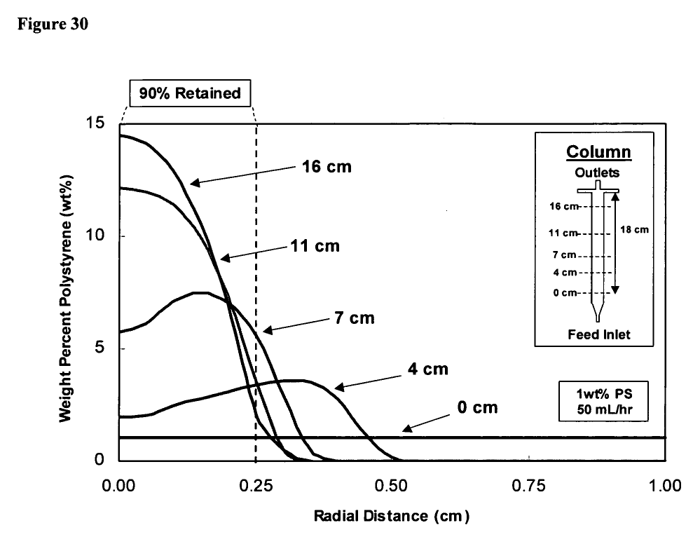

FIG. 31 depicts a complete quadrupole system, including the magnet assembly, the aluminum column, the peristaltic pump, the tubing, and the valves. A beaker was used to hold the feed and glass vials were used to collect the samples from the outlets.

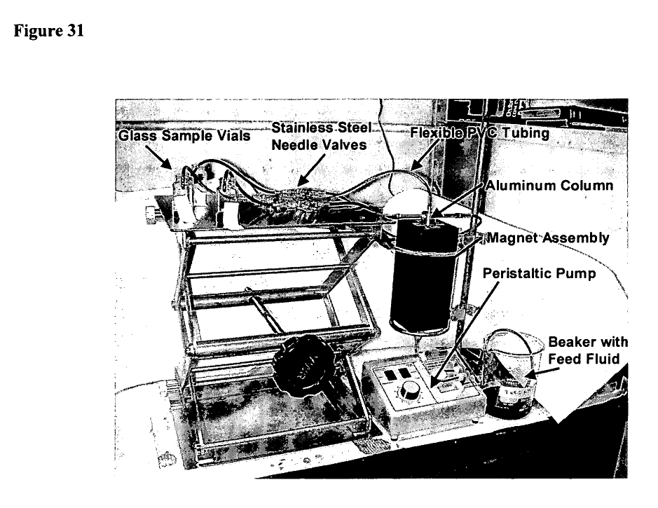

FIG. 32 depicts the force balance on a non-magnetic particle in the counter-current device.

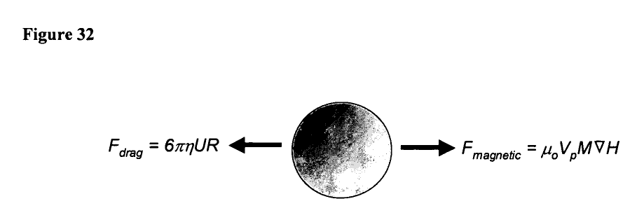

FIG. 33 depicts the flow pattern in the quadrupole column associated with a 20% flow rate for the central outlet.

FIG. 34 depicts the oncentration profile of the polystyrene content in the quadrupole outlets for a control experiment using 160 mL of 1 wt % polystyrene as the feed with no magnetic fluid present in the system. Clarified Feed represents the average concentration of the polystyrene collected at specific time intervals from the two side outlet streams, and Collection Outlet represents the polystyrene concentration collected at specific time intervals from the central outlet stream.

FIG. 35 depicts results for the control experiment using 150 mL of 1 wt % magnetic fluid as the feed, with 1 wt % magnetic fluid present in the device at the start of the experiment. Clarified Feed represents the average magnetite concentration and nanoparticle size collected from the two side outlet streams; Collection Outlet represents the magnetite concentration and nanoparticle size collected from the central outlet stream; Feed represents the magnetite concentration and nanoparticle size of the feed fluid; and Retained represents the magnetite concentration and nanoparticle size of the fluid retained in the column at the end of the experiment.

FIG. 36 depicts the volume-average distribution of the hydrodynamic diameter of magnetic particles in the feed, the outlets, and the fluid retained in the column at the end of the control experiment. Feed represents the feed fluid; Clarified Feed represents the fluid collected from the two side outlet streams; Collection Outlet represents the fluid collected from the central outlet stream; and Retained in Column represents the fluid retained in the device at the end of the experiment; 36(a) depicts the full curves; and 36(b) shows detail from the front half of the curves.

FIG. 37 depicts the concentration profile of the polystyrene content in the quadrupole outlets for 2 micron polystyrene beads using 160 mL of 1 wt % polystyrene and 1 wt % magnetic fluid as the feed with 1 wt % magnetic fluid present in the system. Clarified Feed represents the average concentration of the polystyrene collected at specific time intervals from the two side outlet streams; and Collection Outlet represents the polystyrene concentration collected at specific time intervals from the central outlet stream.

FIG. 38 is a photograph of the inlet section of the aluminum column in the magnet assembly.

FIG. 39 depicts a contour plot showing the magnetic flux density (B) along an axial cross section of the quadrupole column and magnets. The axial magnetic flux density gradient extends approximately a quarter centimeter on either side of the magnet edges through the column. The units are Tesla (T).

FIG. 40 is a graph of axial field lines for the magnetic flux density at different points along the radius of the column; r=1 corresponds to the column walls; and r=0 corresponds to the column centerline. The dashed line at an axial distance of 1 cm corresponds to the edges of the magnets or the entrance into the magnet assembly.

FIG. 41 depicts velocity field profiles for 2 micron particles at different flow rates: (a) 2 mL/hr; (b) 10 mL/hr; and (c) 30 mL/hr. The thick dashed line at an axial distance of 1 cm corresponds to the edges of the magnets or the entrance into the magnet assembly. The velocity field profile is unchanged from approximately 2 cm (1 cm after entry into the magnet assembly) to the top of the column, shown up to 3 cm. The arrows representing the fluid velocity have been normalized as V/Vₘₐₓ, where vₘₐₓ is the maximum linear velocity of the fluid through the column.

FIG. 42 depicts particle trajectories at different flow rates: (a) 2 mL/hr (axial scale changed to enhance detail); (b) 30 mL/hr; (c) 120 mL/hr; and (d) 240 mL/hr. The thick dashed lines represent position in the column at constant time. The dashed line at an axial distance of 1 cm corresponds to the edges of the magnets or the entrance into the magnet assembly. The dashed line at a radial position of 0.25 cm corresponds to the position of the coaxial inner cylinder at the top of the column.

FIG. 43 depicts particle trajectories at 2 mL/hr: (a) calculated with the presence of the axial and radial magnetic field gradients at the entrance to the magnet assembly; (b) calculated in the absence of the axial gradients but in the presence of the radial gradients at the entrance; and (c) calculated in the absence of both the axial and radial gradients at the entrance to the magnet assembly. The thick dashed lines represent position in the column at constant time. The dashed line at an axial distance of 1 cm corresponds to the edges of the magnets or the entrance into the magnet assembly. The dashed line at a radial position of 0.25 cm corresponds to the position of the coaxial inner cylinder at the top of the column.

FIG. 44 depicts the concentration profile of the polystyrene content in the quadrupole outlets for 1.17 micron polystyrene beads using 160 mL of 1 wt % polystyrene and 1 wt % magnetic fluid as the feed with 1 wt % magnetic fluid present in the system. Clarified Feed represents the average concentration of the polystyrene collected at specific time intervals from the two side outlet streams; and Collection Outlet represents the polystyrene concentration collected at specific time intervals from the central outlet stream.

FIG. 45 depicts the percent of polystyrene beads removed from the feed fluid versus feed flow rate for 1 and 2 micron polystyrene beads, using 1 wt % polystyrene and 1 wt % magnetic fluid for the feed.

FIG. 46 depicts the concentration of 2 micron non-magnetic particles as a function of radial distance in the column for different \tilde{Ψ}² and \tilde{β} values, shown at a constant axial distance of 16 cm up the column, prior to reaching the central outlet.

FIG. 47 depicts the percentage of polystyrene beads in the central outlet versus feed flow rate for 1 and 2 micron polystyrene beads using 1 wt % polystyrene and 1 wt % magnetic fluid; the model predictions are presented as dark solid lines. Circles represent 1 micron experimental results and squares represent experimental results using 2 micron polystyrene beads.

FIG. 48 depicts the concentration profile of the cell content in the quadrupole outlets for E. coli cells using 160 mL of 0.4 wt % cells and 1 wt % magnetic fluid as the feed with 1 wt % magnetic fluid present in the system. Clarified Feed represents the average concentration of the cells collected at specific time intervals from the two side outlet streams; and Collection Outlet represents the cell concentration collected at specific time intervals from the central outlet stream.

FIG. 49 depicts the trajectories for 12 micron cell aggregates at different radial locations in the quadrupole column at a flow rate of 500 mL/hr. The thick dashed lines represent position in the column at constant time. The dashed line at an axial distance of 1 cm corresponds to the edges of the magnets or the entrance into the magnet assembly. The dashed line at a radial position of 0.25 cm corresponds to the position of the coaxial inner cylinder at the top of the column.

### DETAILED DESCRIPTION OF THE INVENTION

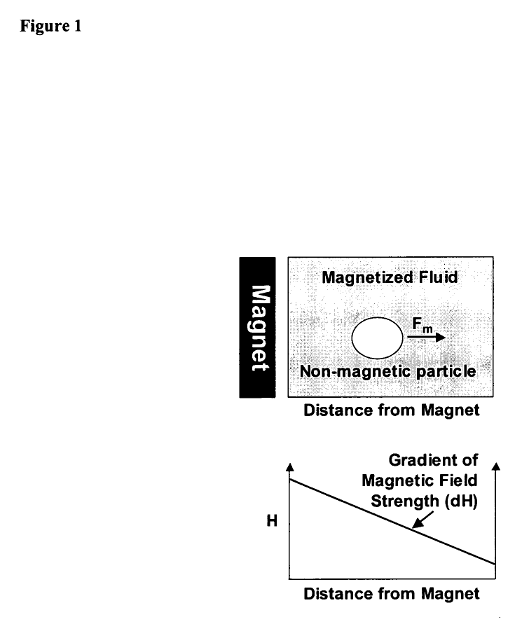

The present invention provides devices and methods for magnetophoretic cell clarification. In the present invention, cells can be clarified from fermentation broth using a method in which the cells are mixed with a magnetic fluid. Cells, which are non-magnetic particles, can be separated from the mixture of fermentation broth and magnetic fluid due to the force that a non-magnetic particle feels when surrounded by a magnetic fluid in the presence of a degrading magnetic field (FIG. 1). The force is described by the following equation:
Fₘ=μ₀VₚΔM·∇H
where μ₀ is the permeability of free space, Vₚ is the volume of the non-magnetic particle, ΔM is the difference between the magnetization of the particle and the magnetic fluid, and H is the magnetic field. The magnetizatoin of the magnetic fluid forces a non-magnetic particle that is surrounded by the magnetic fluid away from areas of high magnetic field strength and into areas of low magnetic field strength. Using this method, non-magnetic particles can be focused and moved out of the bulk fluid by proper design of the magnetic field, the magnetic field gradient, and the geometry of the system, thus leading to magnetophoretic cell clarification.

Magnetic fluids offer several advantages for cell separation. Magnetophoretic cell clarification is size dependent, with larger non-magnetic particles or cells experiencing a stronger force from the magnetization of the surrounding magnetic fluid. This size-dependent force can be applied to a bulk fluid mixture of cells and magnetic fluid. As a result, magnetophoretic cell clarification is not prone to fouling or clogging, which is often a concern in membrane filtration. Magnetophoretic cell clarification also requires no high-speed moving parts and is very gentle on cells, unlike centrifugation. Remarkably, magnetophoretic cell clarification is gentle enough on cells that it may be used when the cells themselves are the product of interest from the bulk fluid.

### DEFINITIONS

For convenience, before further description of the present invention, certain terms employed in the specification, examples, and appended claims are collected here.

The term “cell clarification” as used herein refers to the process of separating cells (bacteria, cultured cells, yeast, fungi, etc.) from fermentation broth, culture medium, or growth medium. Known cell clarification techniques include centrifugation and membrane filtration.

The terms “cultured mammalian cells” and “mammalian cells” and the like, refer to cell lines derived from mammals that are capable of growth and survival when placed in either monolayer culture or in suspension culture in a medium containing the appropriate nutrients and growth factors.

The term “chemical coprecipitation” as used herein refers to a common technique for making aqueous magnetic fluids from metal salts. This technique may be used to produce ferrite particles, such as magnetite (Fe₃O₄), maghemite (γ-Fe₂O₃), or cobalt ferrite (CoFe₂O₄).

The term “diamagnetic” as used herein means having a negative magnetic susceptibility.

The term “fermentation” as used herein refers to the process of culturing cells or other microorganisms in a container, bioreactor, or fermenter.

The term “graft copolymer” as used herein refers to a polymer in which the main backbone of molecular units has side chains attached at various points. The side chains contain different molecular units than the main chain.

The term “magnet” as used herein refers to a substance composed of ferromagnetic or ferrimagnetic material having domains that are aligned to produce a net magnetic field outside the substance or to experience a torque when placed in an external magnetic field.

The term “magnetic core” as used herein refers to a piece of magnetic material, often of iron oxide or ferrite that is within a copolymer shell, coil, transformer, or electromagnet.

The term “magnetic field” as used herein refers to a vector field occupying physical space wherein magnetic forces maybe detected, typically in the presence of a permanent magnet, current-carrying conductor, or an electromagnetic wave.

The term “magnetic field strength” as used herein refers to a vector field used to describe magnetic phenomena, having the property that the curl of the field is equal to the free current density vector in the meter-kilogram-second system of units.

The terms “magnetic fluid” or “ferrofluid” as used herein refers to a fluid or suspension composed of solid magnetic particles of subdomain size colloidally dispersed in a liquid carrier.

The term “magnetic separation” as used herein refers to a process that uses a magnetic solid and an external magnetic field to separate materials or compounds. Examples of magnetic separation include magnetocollection, magnetoflocculation, and magnetoanisotropic sorting.

The term “magnetic susceptibility” as used herein refers to the ratio of the magnetization of a substance to the applied magnetic field strength.

The term “magnetite” as used herein refers to a chemical compound represented as Fe₂O₃*FeO or Fe₃O₄ in the spinel iron oxide species with a 2:1 molar ratio of Fe ions that are present in their III and II oxidation states, respectively.

The term “magnetite nanoparticles” as used herein refers to either the magnetic core of the magnetic particles that make up a magnetic fluid, or to the magnetic particles as a whole, including both the magnetite core and the polymer shell that stabilizes them in the surrounding liquid.

The term “non-magnetic particle” as used herein refers to a particle that does not have inherent magnetic properties.

The term “superparamagnetism” as used herein refers to the tendency of fine particles to behave independently of one another in a manner similar to paramagnets, so that the particles show a net magnetization in the presence of a magnetic field, but then rapidly relax to show zero net magnetization when the applied magnetic field is removed.

The term “transformed cells” as used herein refers to cells and cultures or lines derived therefrom without regard for the number of transfers. It is understood that all the progeny may not be precisely identical in DNA content.

Structure of Magnetic Fluids

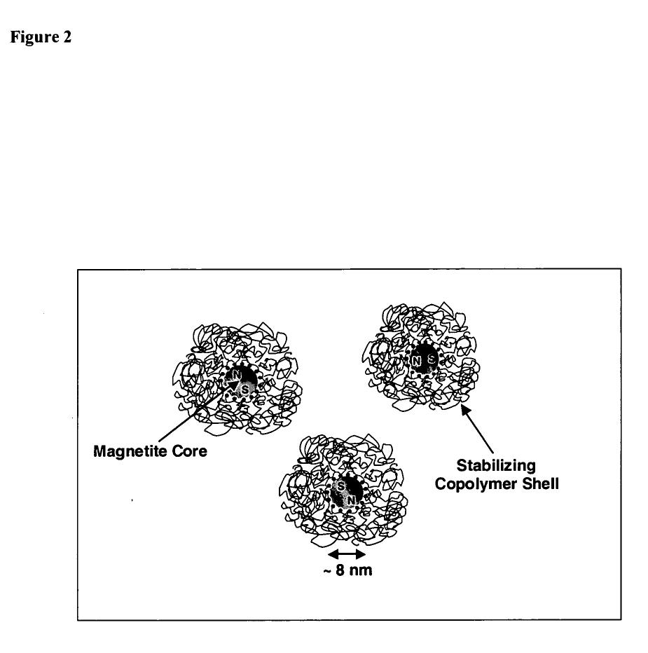

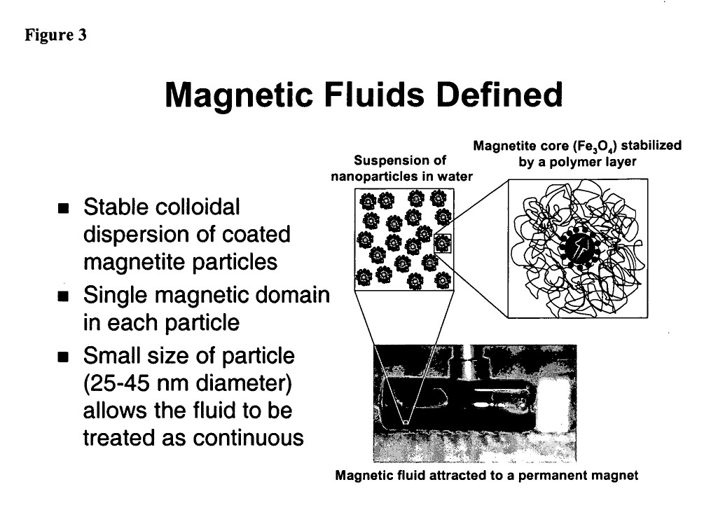

The magnetic fluids used in the present invention are colloidal dispersions of magnetic nanoparticles suspended in a carrier fluid that due to their small size, do not settle under the influence of either gravity or moderate magnetic fields. Further, the magnetic fluids synthesized and used in this invention do not aggregate while in solution due to the presence of a polymer surface coating that is added to the magnetic core during synthesis. The structure of such a magnetic fluid is shown schematically in FIGS. 2 and 3.

Magnetic fluid compositions may include, but are not limited to, metals such as iron, cobalt, nickel, chromium, titanium, manganese, aluminum, copper, samarium, neodynium and the like; metal alloys such as iron-nickel, iron-cobalt, iron-copper, iron-cobalt-aluminum and the like; and raw oxide particles such as magnetite, cobalt ferrite, nickel ferrite and the like.

In the present invention, magnetic fluids comprise magnetite nanoparticles composed of the ferrimagnetic material, magnetite (Fe₂O₃*FeO). This compound is a spinel iron oxide species with a 2:1 molar ratio of Fe ions in their III and II oxidation states, respectively (Gokon et al., Magn. Magn. Mater. 2002; 238:47-55). Use of magnetite, which is not prone to oxidation, is advantageous over magnetic fluids based on cobalt or other iron nanoparticles, which tend to lose their magnetic properties over time (Pathmamanoharan et al., J. Colloid Interface Sci 1998; 205:340-353). The typical magnetite particle size is approximately 8-10 nm, which is sufficiently small to prevent sedimentation of the particles, as Brownian motion for particles of this size will dominate both the gravitational force and the magnetic force that is produced from a typical handheld magnet (Rosensweig, R. E., Ferrohydrodynamics; Dover Publications, Inc.: Mineola, N.Y., 1985).

Without a stabilizing layer surrounding the magnetite core, the magnetite nanoparticles in a magnetic fluid would rapidly flocculate and settle out of solution due to the van der Waals attractive force that exists between them. For a moderately magnetic material like magnetite, the van der Waals force is more important than interparticle magnetic attraction at short range (Shen et al., Langmuir 2001; 17:288-299.) Thus, the role of the stabilizing polymer layer is to prevent flocculation by exerting a repulsive force between the magnetic particles at short range.

In general, magnetic nanoparticles are dispersed in a carrier fluid, which may be a protic or aprotic solvent. Suitable protic solvents include water, alcohols, phenols, amines, and acids. In certain instances, the alcohol is methanol, ethanol, or propanol. Alternatively, the reactions may be run in an aprotic solvent, preferably one in which the reaction ingredients are substantially soluble. Suitable solvents include ethers such as diethyl ether, 1,2-dimethoxyethane, diglyme, t-butyl methyl ether, tetrahydrofuran and the like; siloxanes such as dimethylsiloxane, polyakylsiloxane, polydimethylsiloxane, polyfluropropylmethylsiloxane and the like; halogenated solvents such as chloroform, dichloromethane, dichloroethane, chlorobenzene, carbon tetrachloride, and the like; aliphatic or aromatic hydrocarbon solvents such as benzene, xylene, toluene, hexane, pentane and the like; esters and ketones such as ethyl acetate, acetone, and 2-butanone; polar aprotic solvents such as acetonitrile, dimethylsulfoxide, dimethylformamide, pyridine, and the like; or combinations of two or more solvents.

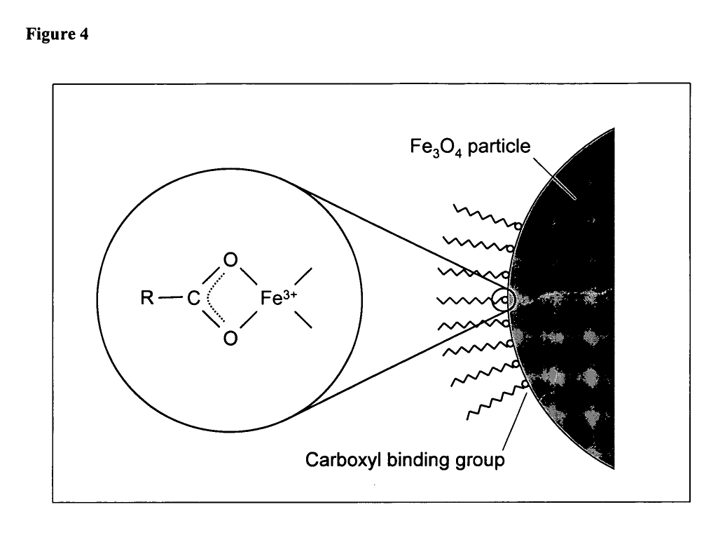

In the present invention, the magnetic particles were suspended in water and graft copolymers were used to prevent the particles from agglomerating. These graft copolymers were chosen so that the polymer was sufficiently solvated by water to induce repulsive interactions when the stabilizing layers of two magnetic particles overlapped. The graft copolymer was attached to the magnetite particle itself through the use of a carboxylic acid functional group, which is known to form a strong d-orbital chelation to iron atoms on the magnetite surface, as shown in FIG. 4 (Mikhailik et al. Colloids Surf. 1991; 52:315-324). This method of attachment coupled with the use of alkyl group steric stabilization was employed in the earliest magnetic fluids (U.S. Pat. No. 3,215,572; Reimers and Khalafalla “Preparing Magnetic Fluids by a Peptizing Methods,” Twin Cities Metallurgy Research Center, U.S. Department of the Interior, 1972), which consisted of fatty-acid stabilized magnetite nanoparticles in kerosene, where the carboxyl head group of the fatty acid attached to the magnetite surface and the alkyl tail provided steric stabilization in the kerosene medium.

In one embodiment of the invention, graft copolymers comprise a polymeric subunit, for example, but not limited to synthetic polymers such as polyphosphazines, poly(vinyl alcohols), polyamides, polycarbonates, polyalkylenes, polyacrylamides, polyanhydrides, poly(phosphoesters), polyalkylene glycols, polyalkylene oxides, polyalkylene terephthalates, polyvinyl ethers, polyvinyl esters, polyvinyl halides, polyvinylpyrrolidone (PVP), polyglycolides, polysiloxanes, polyphosphates, polyurethanes and the like; synthetically modified natural polymers such as alkyl celluloses, hydroxyalkyl celluloses, cellulose ethers, cellulose esters, nitrocelluloses, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate cellulose sulfate sodium salt, and the like; biodegradable polymers such as polylactide, polyglycolide, polycaprolactone, polycarbonate, poly(phosphoesters), polyanhydride, polyorthoesters, and the like; and natural polymers such as a lginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins.

In the present invention, PAA-PEO/PPO (polyacrylic acid-polyethylene oxide/polypropylene oxide) amphiphilic graft copolymers were used to stabilize the magnetite nanoparticle. In certain embodiments, the PEO/PPO ratio is between about 90/10 to about 10/90. In a certain embodiment, the PEO/PPO ratio is 70/30.

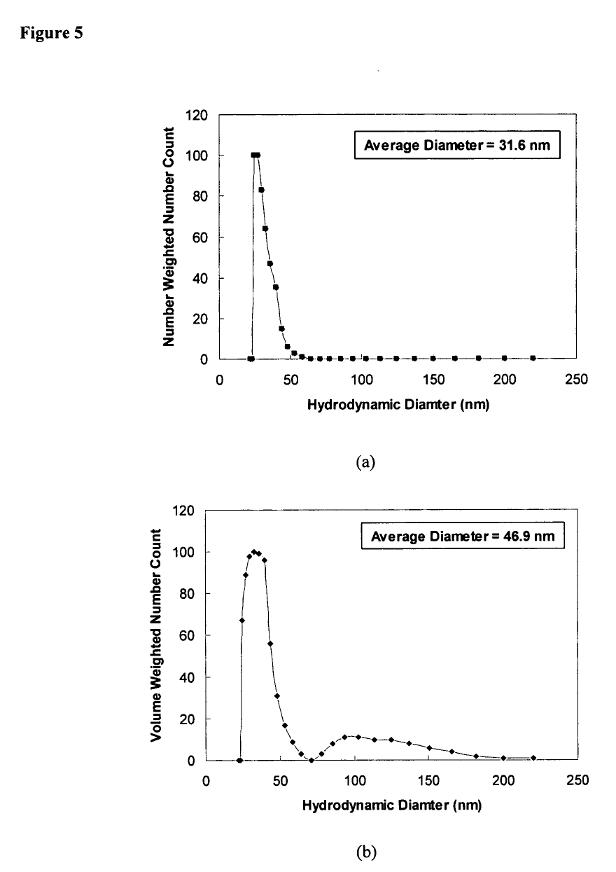

The magnetite nanoparticles comprised by magnetic fluids may be sufficiently small to be single domain particles. The domain size of magnetite is approximately 25 nm (Lee et al., J. Colloid Interface Sci. 1996; 177:490-494), which implies that the about 8-10 nm particles are composed of a single crystal of magnetite, each having a permanent magnetic dipole, similar to that of the bulk material (FIG. 5). In a magnetic fluid, these dipoles are randomized due to either Brownian relaxation (particle rotation) or Neel relaxation (spontaneous fluctuation of the dipole direction within the particle). The dominant mechanism depends on the size of the particle (Rosensweig, R. E., Ferrohydrodynamics; Dover Publications, Inc.: Mineola, N.Y., 1985).

Magnetic fluids exhibit superparamagnetism, such that they have approximately zero net magnetization in the absence of an applied magnetic field, but become strongly magnetized in the presence of an applied field. This magnetization is due to the alignment of the magnetite nanoparticle dipoles with the direction of the applied magnetic field.

In one aspect of this invention, the small size of the magnetic particles allows the magnetic fluid to be treated as a continuum when mixed with cells. In one embodiment, the cell size is about 0.1-100 μm. In further embodiments, the cell size is about 1-50 μm. In a still further embodiment, the cell size is about 1-10 μm. In certain embodiments, the average cell size is about 2 μm.

Magnetic Fluid Synthesis

The synthesis of the magnetic fluid used in this invention involved two steps that were performed in tandem: the formation of the magnetite nanoparticles and the coating of the nanoparticles with the stabilizing graft copolymer layer. The synthesis of the magnetite nanoparticles was conducted in the presence of the graft copolymer to prevent agglomeration of the magnetite particles as they nucleated, in addition to providing long-term stability of the particles. The technique to create the magnetic fluid used in this invention, called chemical coprecipitation, is discussed in detail below.

Chemical coprecipitation, a technique that is well suited for making aqueous magnetic fluids from metal salts, was first achieved by Reimers and Khallafalla (Reimers, G. W.; Khalafalla, S. E. “Preparing Magnetic Fluids by a Peptizing Method,” Twin Cities Metallurgy Research Center, U.S. Department of the Interior, 197). This technique may be used to produce ferrite particles, such as magnetite (Fe₃O₄) (Reimers, G. W.; Khalafalla, S. E. “Preparing Magnetic Fluids by a Peptizing Method,” Twin Cities Metallurgy Research Center, U.S. Department of the Interior, 1972), maghemite (γ-Fe₂O₃) (Massart, R. et al., J. Magn. Magn. Mater. 1995, 149, 1-5), or cobalt ferrite (CoFe₂O₄) (Giri, A. K. et al., IEEE Trans. Magn. 2000, 36, 3029-3031) and is the most common method for preparing magnetic fluids, due primarily to its simplicity and relatively low cost. The technique used to create magnetite nanoparticles in aqueous solution is further described below.

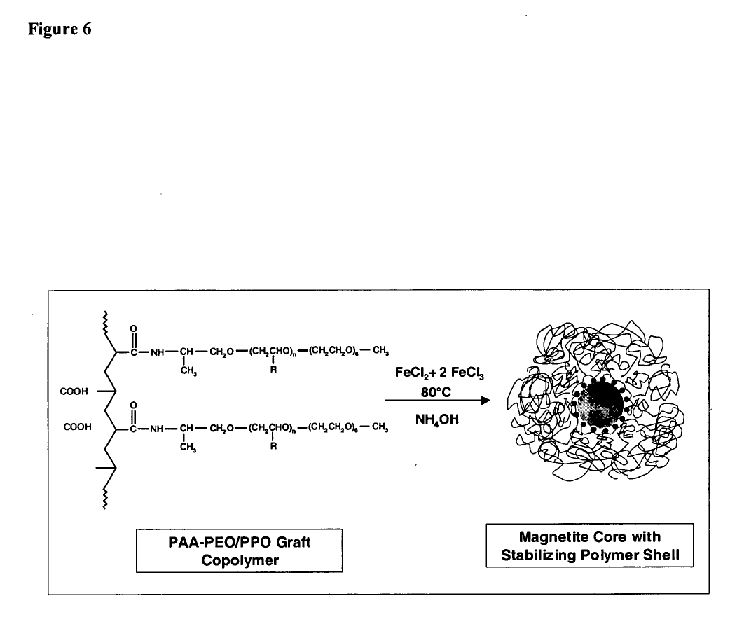

Magnetite as a bulk metal is formed by the basic precipitation of an aqueous solution of iron (III) chloride and iron (II) chloride in a 2:1 molar ratio, forming a spinel structure of Fe³⁺ and Fe²⁺ ions that results in a net magnetic dipole (Gokon, N. et al, J. Magn. Magn. Mater. 2002, 238, 47-55). Magnetite nanoparticles are formed when this reaction is conducted in the presence of a dissolved graft copolymer that binds to the magnetite particles just after nucleation, thus limiting their growth to approximately 8-10 nm (FIG. 6). The overall stoichiometry of this reaction is shown below, with ammonium hydroxide serving as the precipitating agent.
2 FeCl₃+FeCl₂+8 NH₄OH→Fe₃O₄+8 NH₄Cl+4 H₂O

The ammonium hydroxide is added in excess so that the pH of the aqueous reaction medium is strongly basic (pH of 12-14). The size, composition, and magnetization of the resultant nanoparticles are affected by the reagent concentrations, the graft copolymer concentration, the temperature, and the pH during synthesis (Feltin, N. and Pileni, M. P. Langmuir 1997, 13, 3927-3933; Blums, E. et al., Magnetic Fluids; Walter de Gruyter and Co.: Berlin, Germany, 1996; U.S. Pat. No. 4,094,804; Shen, L. et al., Langmuir 1999, 15, 447-453; Bica, D. Romanian Rep. Phys. 1995, 47, 265-272). The optimal reaction temperature for the formation of magnetite is generally thought to be approximately 80° C. (Shen, L et al., Langmuir 1999, 15, 447-453; Bica, D. Romanian Rep. Phys. 1995, 47, 265-272); and this temperature was used for the formation of the magnetic fluid used in this invention.

Magnetophoretic Separation Using Magnetic Fluids

In magnetophoretic separation, a magnetic fluid is used to exert forces on non-magnetic particles in order to separate them on the basis of their size. This approach is distinct from the biological separations discussed above, as it utilizes the magnetic fluid for its magnetic properties only, i.e., not for any functionalized surface properties.

On the other hand, magnetoflotation has been used to separate coal particles of different densities by suspending the particles in a magnetic fluid and applying a vertical magnetic field gradient (Fofana, M et al., Miner. Metall. Process. 1997, 14, 35-40). The field gradient causes the particles to experience a body force that acts opposite to gravity, changing the effective density of the fluid. By changing the magnetic field gradient, the effective fluid density can be set between the density of two types of particles, causing one to float and the other to sink (Fofana, M. et al., Miner. Metall. Process. 1997, 14, 35-40).

Remarkably, we have discovered that magnetophoretic cell clarification can be applied to all cell separations. By suspending cells in a magnetic fluid, the cells can be driven against a magnetic field gradient. Transport of cells is opposed by the drag force on the cells, allowing sorting based on the cell size.

Magnetophoretic Cell Clarification Using a Counter-Current Device

The concept of magnetophoretic cell clarification was first tested using a counter-current device. In a counter-current device, pairs of magnets may move in a direction opposite to the flow of the feed mixture. The motion of the magnetic field produced by the moving magnets captures the cells from the feed mixture and pushes them into a separate collection tube, resulting in clarified bulk fluid that then exits the counter-current device. A counter-current device comprises a plurality of pairs of magnets, tubing, a chain that is rotated by a motor, a syringe pump, a feed inlet, and a clarified feed outflow.

A counter-current device comprises a series of magnets that are mounted to a chain which is rotated by a motor. Magnets are mounted on a chain in pairs that face each other, but are separated by an opening. In certain embodiments, a counter-current device comprises greater than about 2, but less than about 100 magnet pairs. In further embodiments, a counter-current device comprises greater than about 10, but less than about 50 magnet pairs. In a further embodiment, a counter-current device comprises approximately 36 magnet pairs.

In certain embodiments, the distance separating each pair of magnets mounted on a chain is about 1-100 mm. In further embodiments, the distance separating each pair of magnets mounted on a chain is about 10-50 mm. In a further embodiment, the distance separating each pair of magnets mounted on a chain is approximately 12.7 mm.

In certain embodiments, the magnets used in a counter-current device are permanent magnets or electromagnets. In further embodiments, the magnets are permanent magnets. In a further embodiment, the magnets used in a counter-current device are non-rare earth permanent magnets such as Alnico (Aluminum-Nickel-Cobalt) magnets and Ceramic (Strontium and Barium Ferrite) magnets. In a further embodiment, the magnets used in a counter-current device are rare earth magnets such as Samarium-Cobalt magnets and Neodymium-Iron-Boron magnets. In a still further embodiment, the magnets used in a counter-current device are Neodymium-Iron-Boron 39 MGOe magnets. The dimensions of each magnet may range from about 3×3×3 mm to about 300×200×100 mm. In certain embodiments, the dimensions of each magnet are approximately 11.7×11.7×5 mm.

In certain embodiments, the chain used to mount the pairs of magnets is rotating. In a further embodiment, the chain is elliptical in shape. In certain embodiments, the length of the chain may range from about 20-300 cm. In further embodiments, the length of the chain is between about 50-150 cm. In certain embodiments, the length of the chain is approximately 90 cm.

The movement of the magnets mounted on a rotating chain is generated by a motor. In certain embodiments, the speed of the magnets on a rotating chain is kept constant. In further embodiments, the speed of the magnets on a rotating chain is between about 1 and about 10 cm/min. In a further embodiment, the speed of the magnets on a rotating chain is approximately 5.3 cm/min.

In certain embodiments, the opening that separates the pair of magnets in a counter-current device is about 2.5-65 mm. In further embodiments, the opening separating the pairs of magnets is about 3-20 mm. In a further embodiment, the opening separating the pairs of magnets in a counter-current device is about 3.2 mm.

The spacing between the pairs of magnets in a counter-current device is to accommodate flow tubing through which the feed mixture is pumped between the magnet pairs. In certain embodiments, the flow tubing is clear PVC tubing about 1/32″ (0.79 mm) ID and 3/32″ (2.38 mm) OD to about 2″ (50.8 mm) ID and 2½″ (63.5 mm) OD. In further embodiments, the flow tubing is Nalgene 180 Clear PVC tubing about 1/16″ (1.59 mm) ID and about ⅛″ (3.18 mm) OD.

In certain embodiments, the length of the flow tubing may range from about 10-100 cm. In further embodiments, the length of the flow tubing is between about 40-80 cm. In certain embodiments, the length of the flow tubing is approximately 60 cm.

The feed mixture may be introduced into a counter-current device through a T-junction located at a bottom corner of the device and may be pumped through the tubing with the use of a syringe pump. In one embodiment, the syringe pump is a Sage Syringe Pump Model M365.

Once pumped into the T-junction, the feed flows from the T-junction in a direction opposite of magnet movement, around the rotating chain, and exits at a side opposite from the feed inlet. In certain embodiments, the T-junction for the feed inlet is located at the bottom right corner of the device. In this embodiment, feed flows to the right of the T-junction, up around the top of the apparatus, and exits at the lower left side of the device.

In certain embodiments, the tube to the left of the T-junction is closed at the distal end and experiences no real fluid movement, but instead serves as a collection reservoir for the non-magnetic particles in the feed. The collection tube is filled with pure magnetic fluid at the start of each experiment. In certain embodiments, the collection tube is about 1-100 cm in length. In further embodiments the collection tubing is about 1-50 cm in length. In certain embodiments, the collection tubing is approximately 41 cm in length.

In certain embodiments, the present invention relates to a counter-current device comprising an elliptical rotating chain with about 36 pairs of magnets mounted onto said chain with a distance of about 12.7 mm between each magnet pair and separated by an opening equal to about 3.2 mm.

In further embodiments, the present invention relates to a counter-current device comprising an elliptical rotating chain with about 36 pairs of magnets mounted onto said chain with a distance of about 12.7 mm between each magnet pair and separated by an opening equal to about 3.2 mm, which contains flow tubing used to pump the feed mixture between the magnet pairs.

In still further embodiments, the present invention relates to a counter-current device comprising an elliptical rotating chain with about 36 pairs of magnets mounted onto said chain with a distance of about 12.7 mm between each magnet pair and separated by an opening equal to about 3.2 mm, which contains flow tubing used to pump the feed mixture between the magnet pairs, and which are rotated at a constant speed of about 5.3 cm/min, collecting the non-magnetic particles into a collection tube.

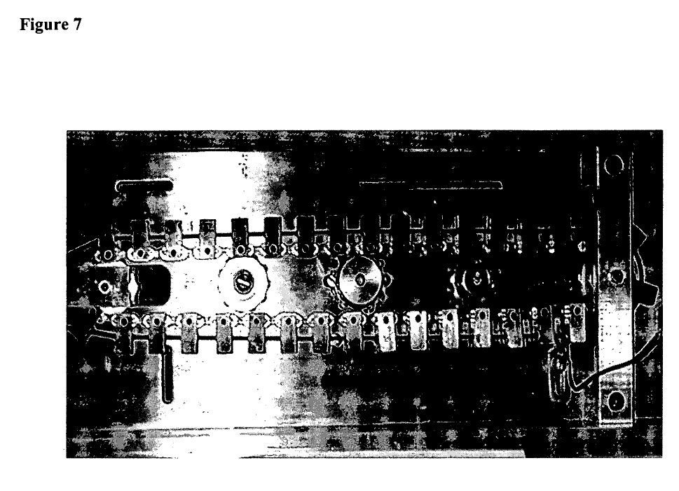

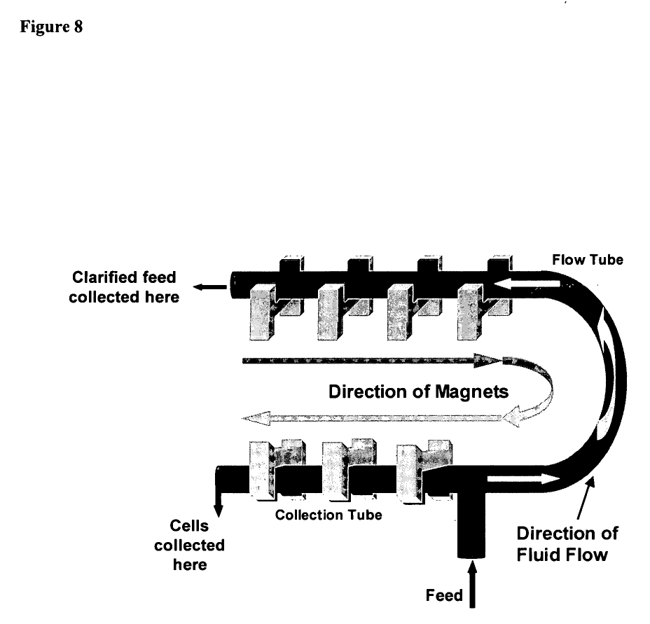

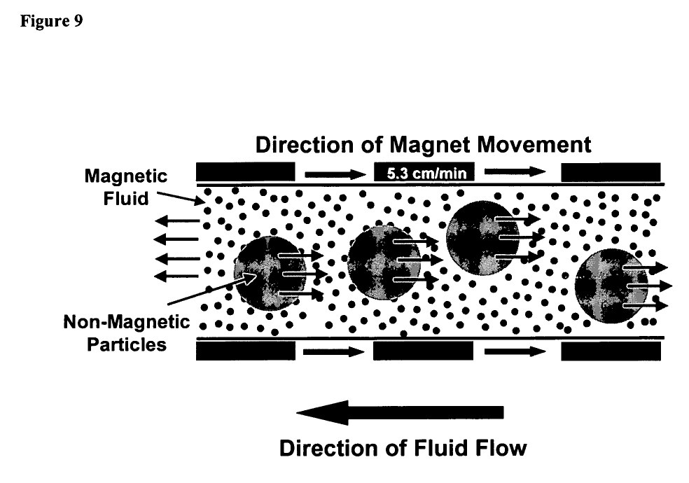

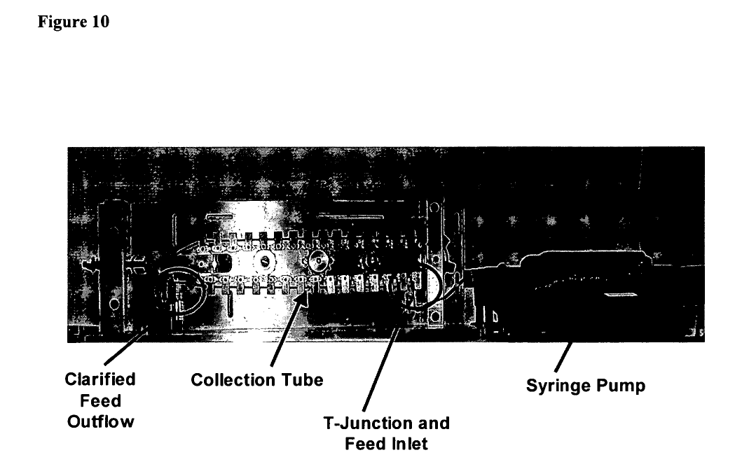

FIG. 7 shows a photograph of magnet pairs mounted on a rotating chain of a counter-current device of the present invention. A schematic diagram indicating the direction of magnet movement and fluid flow for a counter-current device is shown in FIG. 8 and 9. FIG. 10 shows a photograph of a complete counter-current system, including the position of the syringe pump used to pump the fluid through the device.

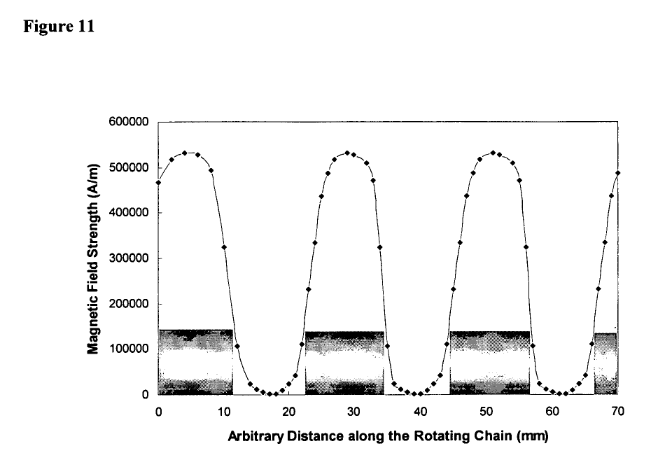

The use of equally spaced pairs of magnets in a counter-current device generates a sinusoidal magnetic field profile along the length of the tubing with a maximum field strength approaching 0.7 Tesla. This sinusoidal field was measured using a Gauss meter and plotted as shown in FIG. 11. The measurements were taken between the two magnets that form the magnet pairs as well as between the empty space between two pairs of magnets, along the same track that the tubing occupies in the device.

A magnetic field profile of the type shown in FIG. 11 forces the cells in the feed to move to areas of weakest magnetic field, in this case between the magnet pairs. As the feed flows through the sinusoidal magnetic field, packets of cells start to form between the magnet pairs. As the magnets move in the direction opposite to the fluid flow, these packets of cells travel with the magnets in order to stay in the areas of weakest field, a nalogous to surfing a magnetic wave. In this way, the cells travel with the magnets against the flow of the bulk fluid, and are directed into the collection tube, where they are sequestered away from the feed fluid.

In other embodiments, the magnets of a counter-current device are not attached to a moving chain. In certain embodiments, the flow channel may be of rectangular cross-section with a varying aspect ration (e.g. 5 mm×50 mm), with magnets of appropriate size on either side of the channel, such that the magnetic field is normal to the thin dimension. The magnets may be attached on rotating drums that move around the channel, which could be in the form of a concentric cylindrical annulus.

In other embodiments, the magnets of a counter-current device are held stationary and the flow channel is rotated within the applied magnetic field. In further embodiments, the magnets and the flow channel of a counter-current device are held stationary, and the feed and outlet points are moved in step increments around the flow channel to simulate the movement of the magnets relative to the flow (e.g. simulated moving beds used in absorption processes).

In other embodiments, a counter-current device may use a series of electromagnets, which can be sequenced to be turned on and off to generate a moving magnetic field without actually moving the magnets themselves.

In still other embodiments, a counter-current device may be operated in either a continuous flow manner, or the channel can be charged with a cell suspension, and the cells moved to the end of the channel through the moving magnetic field.

The present invention encompasses counter-current devices for analytical, benchtop, or industrial use, and which may be used to clarify samples of any volume.

Magnetophoretic Cell Clarification Using a Quadrupole Device

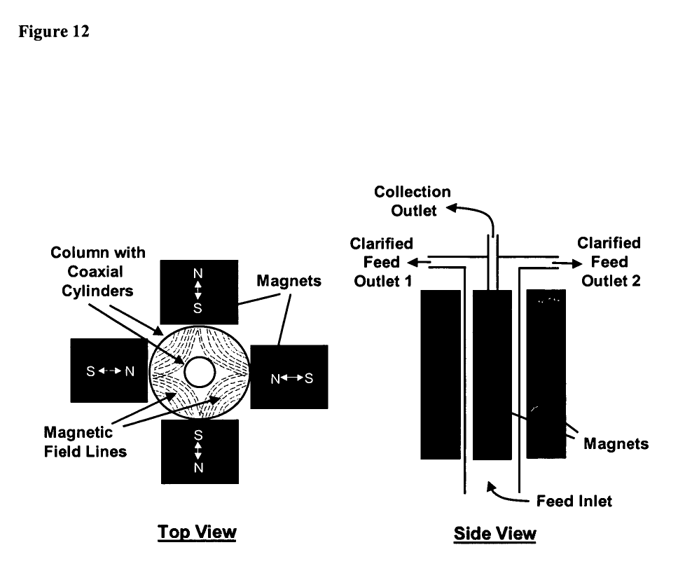

A quadrupole device for magnetophoretic cell clarification was modeled, constructed, and analyzed. The device is called a quadrupole device due to the position of the four magnets used to generate the magnetic field (see FIG. 12).

In a quadrupole device, magnets are arranged around a central cylinder to create areas of high magnetic field along the cylinder wall and areas of low field in the center of the cylinder. Thus, such a device aims to separate non-magnetic particles, e.g. cells, from fermentation broth by focusing them into an area of low magnetic field found in the center of the cylinder, where they will then be collected by a smaller, annular cylinder located at the top of the device, called Outlet 2 in FIG. 12.

A quadrupole device was designed to eliminate moving magnets, to allow for the clarification of larger volumes of feed mixture than was feasible with a counter-current device, and to allow for continuous operation, as opposed to the semi-batch operation of the counter-current device. The design and magnetic field profile of the quadrupole device are discussed in detail below.

The quadrupole device comprises magnets that supply the magnetic field needed for separation and a cylindrical column that is used to pass the feed mixture through the magnetic field.

In certain embodiments, the magnets used in a quadrupole device are permanent magnets or electromagnets. In further embodiments, the magnets used in a quadrupole device are permanent magnets. In a further embodiment, the magnets used in a quadrupole device are non-rare earth permanent magnets such as Alnico (Aluminum-Nickel-Cobalt) magnets and Ceramic (Strontium and Barium Ferrite) magnets. In a still further embodiment, the magnets used in a quadrupole device are rare earth magnets such as Samarium-Cobalt magnets and Neodymium-Iron-Boron magnets. In certain embodiments, the permanent magnets are nickel-plated Neodymium-Iron-Boron 40 MGOe permanent magnets.

In certain embodiments, four permanent magnets are used in a quadrupole device and are arranged in a cross-shape. In further embodiments, the four magnets are arranged equidistant from one another. In other embodiments, similar arrangements of magnets could be achieved using 6, 8, 10, etc. magnets, as long as each magnet is equidistant from each other around the central column.

In certain embodiments, the four magnets are the same size. In further embodiments, the magnets can range from about 3.150″ (8.0 cm) long×0.315″ (0.8 cm) wide×0.315″ (0.8 cm) thick (this is the direction that the magnets are magnetized in) to about 39.370″ (100.0 cm) long×3.937″ (10.0 cm) wide×3.937″ (10.0 cm) thick (this is the direction that the magnets are magnetized in). In certain embodiment, the size of each magnet is about 7.086″ (18.0 cm) long×0.708″ (1.8 cm) wide×0.708″ (1.8 cm) thick (this is the direction that the magnets are magnetized in).

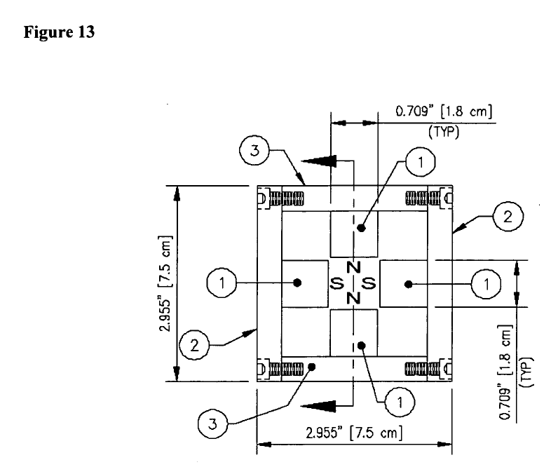

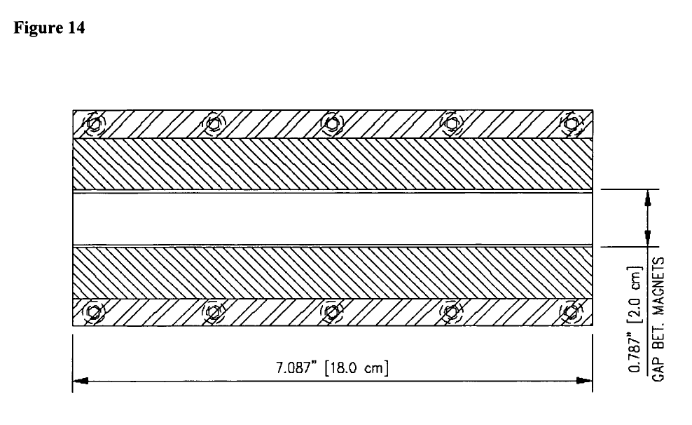

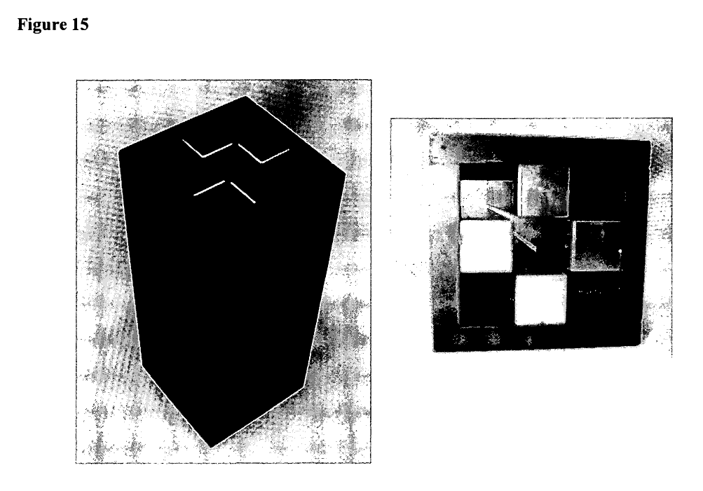

Due to the high magnetic strength of the magnets, they are attached to a stainless steel housing box, which prevents them from snapping together. A schematic diagram of the magnets and a steel housing box is shown in FIGS. 13 and 14. A completed magnet assembly as manufactured by Dura Magnetics, Inc. (Sylvania, Ohio) is shown in FIG. 15.

The orientation of the four magnets in a quadrupole device, as shown in FIG. 13, creates a symmetrical magnetic field that is strongest next to the magnets and weakest in the center of the magnet assembly. The Maxwell 3-D Electromagnetic Field Simulator program, created by Ansoft Corporation, can be used to model the field lines of the magnetic field produced by the alternating north/south, north/south orientation of the four magnets.

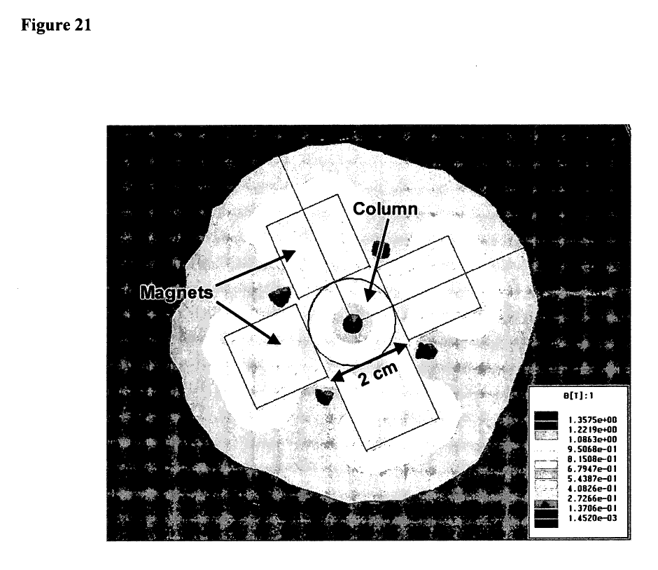

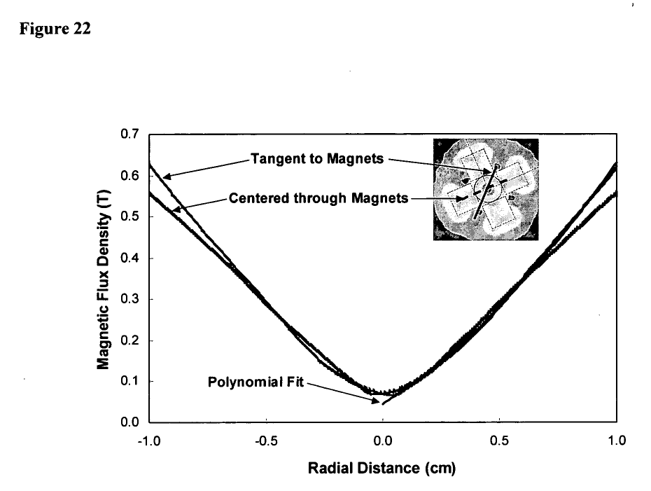

Simulations, as shown in FIGS. 21 and 22, show that the magnetic flux density and thus the magnetic field of a quadrupole device of the present invention is approximately symmetrical. The magnetic flux density is strongest next to the magnets, with a field strength of 0.65 Tesla, and weakest in the center of the magnet assembly, with a field strength of less than 0.1 Tesla. The field gradient, which is how fast the magnetic flux density decays over distance, is about 0.56 Tesla/cm. The simulation was performed using Neodymium-Iron-Boron 35 MGOe, which is actually a slightly weaker magnetic material than the Neodymium-Iron-Boron 40 MGOe used to manufacture the magnets for a quadrupole device of the present invention.

A quadrupole device of the present invention also comprises a cylindrical column to fit inside of the magnet assembly. The purpose of the cylindrical column is to contain the mixture of magnetic fluid and fermentation broth as it moves through the magnetic field created by a quadrupole orientation of the four magnets. Since the magnetic field is weakest in the center of the magnet assembly, the cells in the feed mixture should be induced to move to the center of the column, when the column is placed inside the magnet assembly.

In certain embodiments, the cylindrical column is constructed out of any rigid, non-magnetic material. In further embodiments, the cylindrical column is constructed out of aluminum or plastic. In a certain embodiment, the cylindrical column is constructed out of aluminum. Since aluminum is non-magnetic, the insertion of the column into the magnet assembly should not distort the magnetic field produced by the magnets.

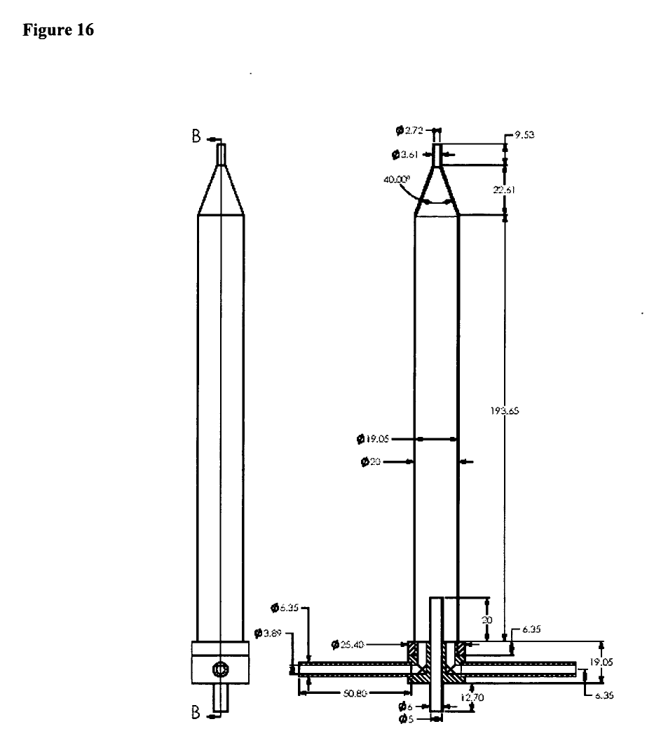

In further embodiments, the cylindrical column comprises an inner cylinder located near the outlet end of the column, from which concentrated cells may be collected and removed from the bulk fluid. In certain embodiments, the cylindrical column comprises an inner cylinder located at the top of the column and a tapered metal tube at the bottom, inlet end of the cylinder. FIG. 16 shows a schematic diagram of a cylindrical column with a tapered inlet connected to the main body of the column and an inner cylinder located near the outlet end of the column.

In certain embodiments, the main body of the cylinder may range from about 10 cm long×1 cm OD to about 115 cm long×12 cm OD, with the inner cylinder ranging from about 1 cm long×0.25 cm ID inside the main body of the cylinder to about 10 cm long×3.0 cm ID inside the main body of the cylinder. The thickness of the cylinder walls can range from about 0.5 mm to about 1 cm. In further embodiments, the main body of the cylinder is about 20 cm long×2 cm OD, with the inner cylinder measuring about 2 cm long×0.50 cm ID inside the main body of the cylinder, and with all main and inner cylinder wall thickness equal to about 0.5 mm. Further dimensions of one embodiment are substantially depicted in FIG. 16.

In certain embodiments, flexible tubing is connected to the tapered inlet end. In further embodiments, the flexible tubing is used to introduce the feed mixture into the cylindrical column. In still further embodiments, the flexible tubing may be about 1/32″ (0.79 mm) ID and 3/32″ (2.38 mm) OD to about 2″ (50.8 mm) ID and 2½″ (63.5 mm) OD. In certain embodiments, the flexible tubing is Nalgene 180 Clear PVC Tubing with the following dimensions about ⅛″ (3.1 mm) ID and 3/16″ (4.76 mm) OD. The inlet tubing may be of any length. In other embodiments, the tubing will not be flexible.

In certain embodiments, flexible tubing is connected to the outlet end of the cylindrical column. The outlet end of the column comprises three outlets, two at the side and one coming from the center of the column. Based on the magnetic field profile of a quadrupole system, the center outlet stream should contain the majority of the cells from the fermentation broth, while the two side outlet streams should contain the clarified bulk fluid. In further embodiments, the flexible tubing may be attached to one or more of the outlets. In a further embodiment, flexible tubing is attached to all three outlets. In a still further embodiment, the flexible tubing is about 1/32″ (0.79 mm) ID and 3/32″ (2.38 mm) OD to about 2″ (50.8 mm) ID and 2½″ (63.5 mm) OD. In certain embodiments, the flexible tubing is Nalgene 180 Clear PVC tubing, about 3/16″ (4.76 mm) ID, 5/16″ (7.94 mm). The outlet tubing may be of any length. In other embodiments, the tubing will not be flexible.

In certain embodiments, a quadrupole device comprises a magnet assembly comprising four permanent magnets oriented in a cross-shape design with each magnet placed equidistant from one another and a cylindrical column placed in the middle of the magnet assembly.

In further embodiments, a quadrupole device comprises a magnet assembly comprising four nickel-plated Neodymium-Iron-Boron 40 MGOe permanent magnets oriented in a cross-shape design with each magnet placed equidistant from one another and a cylindrical column with a tapered inlet and an inner cylinder located near the outlet.

In a further embodiment, a quadrupole device comprises a magnet assembly comprising four nickel-plated Neodymium-Iron-Boron 40 MGOe permanent magnets oriented in a cross-shape design with each magnet placed equidistant from one another and a cylindrical column with a tapered inlet and a inner cylinder located near the outlet with tubing used to introduce the feed mixture and to collect the outlet flows.

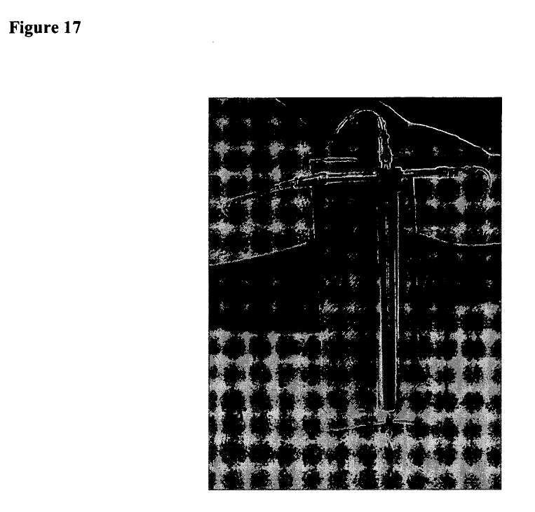

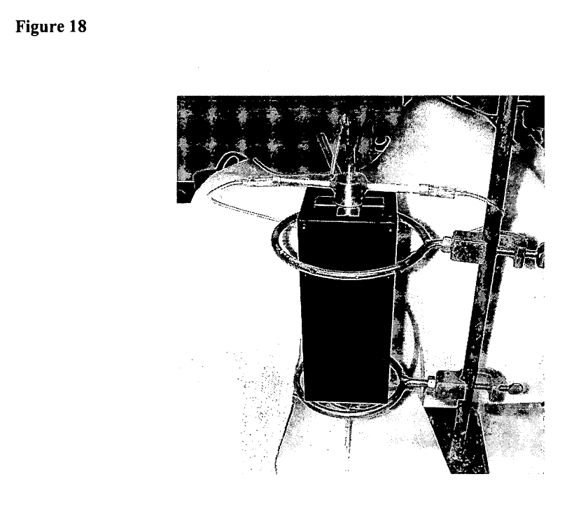

FIG. 17 shows a picture of a cylindrical column and FIG. 18 shows a picture of a quadrupole system, with the cylindrical column sitting in the magnet assembly.

The present invention encompasses quadrupole devices for analytical, benchtop, or industrial use, and which may be used to clarify samples of any volume.

Methods

Using the devices and methods described in the present invention, non-magnetic particles can be separated from bulk fluid using a counter-current or a quadrupole device when said bulk fluid is mixed with magnetic fluid.

In one embodiment of the invention, cells to be separated from a fermentation broth using a magnetic fluid and a counter-current or a quadrupole device are prokaryotic cells including bacterial or cyanobacterial cells. Non-limiting examples of bacteria include a member of the genus Escherichia, Streptococcus, Staphylococcus, Bordetella, Corynebacterium, Mycobacterium, Neisseria, Haemophilus, Actinomycetes, Streptomycetes, Nocardia, Enterobacter, Yersinia, Fancisella, Pasturella, Moraxella, Acinetobacter, Erysipelothrix, Branhamella, Actinobacillus, Streptobacillus, Listeria, Calymmatobacterium, Brucella, Bacillus, Clostridium, Treponema, Salmonella, Kleibsiella, Vibrio, Proteus, Erwinia, Borrelia, Leptospira, Spirillum, Campylobacter, Shigella, Legionella, Pseudomonas, Aeromonas, Rickettsia, Chlamydia, Borrelia and Mycoplasma, and further including, but not limited to, a member of the species or group, Escherichia coli (E. coli), Group A Streptococcus, Group B Streptococcus, Group C Streptococcus, Group D Streptococcus, Group G Streptococcus, Streptococcus pneumoniae, Streptococcus pyogenes, Streptococcus agalactiae, Streptococcus faecalis, Streptococcus faecium, Streptococcus durans, Neisseria gonorrheae, Neisseria meningitidis, Staphylococcus aureus, Staphylococcus epidermidis, Corynebacterium diptheriae, Gardnerella vaginalis, Mycobacterium tuberculosis, Mycobacterium bovis, Mycobacterium ulcerans, Mycobacterium leprae, Actinomyctes israelii, Listeria monocytogenes, Bordetella pertusis, Bordatella parapertusis, Bordetella bronchiseptica, Shigella dysenteriae, Haemophilus influenzae, Haemophilus aegyptius, Haemophilus parainfluenzae, Haemophilus ducreyi, Bordetella, Salmonella typhi, Citrobacter freundii, Proteus mirabilis, Proteus vulgaris, Yersinia pestis, Kleibsiella pneumoniae, Serratia marcessens, Serratia liquefaciens, Vibrio cholera, Shigella dysenterii, Shigella flexneri, Pseudomonas aeruginosa, Franscisella tularensis, Brucella abortis, Bacillus anthracis, Bacillus cereus, Clostridium perfringens, Clostridium tetani, Clostridium botulinum, Treponema pallidum, Rickettsia rickettsii, Helicobacter pylori or Chlamydia trachomitis.

In a further embodiment, the cells to be separated from fermentation broth are bacterial cells including E. coli and B. subtilis strains. In a further embodiment, the cells to be separated from fermentation broth are E. coli cells including DH5, DH5-alpha, JM109, XL-1 Blue, BL21, and INValphaF cells. In a still further embodiment, the cells to be separated from fermentation broth are bacterial cells transformed with a plasmid or recombinant DNA.

In another embodiment, the cells to be separated from culture media or growth media using magnetic fluids and a counter-current or a quadrupole device are eukaryotic cells including cultured mammalian cells, yeast cells and other fungal cells, insect cells, and plant cells.

Examples of cultured mammalian cells may include cells derived from a mammal (e.g. human, mouse, rat) including cells derived from any tissue such as, but not limited to, epithelial cells, fibroblast cells, liver cells, lung cells, endothelial cells, heart cells, blood cells, bone cells, bone marrow cells, pancreatic cells, spleen cells, stomach cells, muscle cells, adrenal cells, kidney cells, urinary bladder cells, cervical cells, ovarian cells, mammary gland cells, uterine cells, placental cells, prostate cells, testicular cells, intestinal cells, colon cells, neural cells, glial cells, B lymphocyte cells, T cells, lymph cells, thymus cells, stem cells, embryonic cells, fetal cells, and carcinoma or tumor cells.

In a further embodiment, the cells to be separated from culture media are cultured mammalian cells including transformed cell lines. Mammalian cell lines may include, but are not limited to, CHO cells, COS cells, MDCK cells, NIH 3T3 cells, HELA cells, Hep G2 cells, L cells, Caco-2 cells, PC12 cells, hybridomas, myelomas, and any derivatives or transformations thereof.

In another embodiment, the cells to be separated from culture media are transformed mammalian cell lines expressing recombinant DNA. In still further embodiments, the cells to be separated from culture media are transformed cell lines expressing recombinant DNA fused with a reporter construct, such as Green Fluorescent Protein (GFP), luciferase, chloramphenical acetyltransferase (CAT), beta-galactosidase.

Additional cultured cell lines may include insect cells, e.g. SF2 cells, and cells derived from plants.

In another embodiment, the cells to be separated from growth media using a magnetic fluid and a device of the present invention are yeast and other fungal cells. Yeast and other fungal cells include, but are not limited, to the genus Acremonium, Alternaria, Amylomyces, Arthoderma, Aspergillus, Aureobasidium, Blastochizomyces, Botrytis, Candida, Cladosporium, Crytococcus, Dictyostelium, Emmonsia, Fusarium, Geomyces, Geotrichum, Issatchenkia, Microsporum, Neurospora, Oidodendro, Paecilomyces, Penicillium, Pilaira, Pityrosporum, Rhizopus, Rhodotorula, Saccharomyces, Stachybotrys, Trichophyton, Trichoporon, and Yarrowia, and further including, but not limited to, a member of the species Acremonium strictum, Alternaria alternata, Amylomyces rouxii, Arthroderma benhamie, Arthroderma cajetani, Arthroderma flavens, Arthroderma gloria, Arthroderma lecticularum, Arthroderma tubercalatum, Aspergillus fumigatas, Aspergillus nidulans, Aspergillus niger, Aspergillus oryzae, Aspergillus terrrus, Candida albicans, Candida glabrata, Candida guilliermondii, Candida kefyr, Candida krusie, Candida lustianie, Candida parapsilosis, Candida tropicalis, Cladosporium herbarum, Cryptococcus albidus, Cryptococcus humicolus, Cryptococcus laeurentii, Cryptococcus neoformans, Cryptococcus uniguttulatus, Dictyostelium discoideum, Neurospora crassa, Oidodendron eschinulatum, Oidodendron griseum, Oidodendron truncatum, Paecilomyces variotto, Penicillium chrysogenum, Penicillium notatum, Pilaira anomal, Piryosporum ovale, Rhizopus oligosporus, Rhizopus stolonifer, Rhodotorula muciloaginosa, Sacharomyces cerevisiae, Sacharomyces pastorianus, Sporothrix schenckii, Stachybotrys chartarum, Trichophyton rubrum, Trichophyton mentagrophytes, Trichosporon cutaneum, and Yarrowia lipolytica.

In further embodiments, the cells to be separated from growth media using a magnetic fluid are the yeast cells, Saccharomyces cerevisiae (S. cerevisiae), also known as Baker's yeast. In a further embodiment, the S. cerevisiae cells to be separated from growth media are expressing mutated copies of one or more gene; deleted for one or more gene; or any combination thereof.

In another embodiment, viral particles may be separated using a magnetic fluid and a counter current or a quadrupole device. Viral particles may be obtained from DNA or RNA virues. Non-limiting examples of DNA viruses include, but are not limited to a member of the family Adenoviridae, Asfaviridae, Ascoviridae, Badnavirus, Baculoviridae, Caulimovirus, Caudoviridae, Ciroviridae, DNA Tumor Viruses, Fuselloviridae, Geminiviridae, Guttaviridae, Hepadnaviridae, Herpersviridae, Inoviridae, Iridoviridae, Lipothrixviridae, Microviridae, Nanovirus, Papillomaviridae, Parvoviridae, Phycodnaviridae, Polydnaviridae, Polyomarviridae, Poxviridae, Rudiviridae, Tectiviridae, and Transfusion-transmitted virus. Non-limiting examples of RNA viruses include, but are not limited to a member of the family Arenaviridae, Astroviridae, Birnaviridae, Bromoviridae, Bunyaviridae, Calicivirdae, Carlaviridae, Closteroviridae, Comorviridae, Coronaviridae, Cystoviridae, Encephalitis viruses, Filoviridae, Flaviviridae, Hepatitis Delta virus, Hepatitis E virus, Leviviridae, Luteovirus, Mononegavirales, Mosaic Viruses, Nidovirales, Nodaviridae, Orthomyxoviridae, Paramyxoviridae, Picobirnavirus, Picornaviridae, Potexvirus, Potyviridae, Reoviridae, Retroviridae, Rhabdoviridae, Sequiviridae, Tenuivirus, Togoviridae, Tombusviridae, and Totiviridae.

In another embodiment, subcellular organelles including, but not limited to, nuclei, mitochondria, lysosomes, perixosomes, chloroplasts, plasma membrane, microsomal fractions (e.g. fragments of endoplasmic reticulum), other membrane bound vesicles, large polyribosomes, small polyribosomes, and ribosomal subunits can be separated using magnetic fluid and a counter-current device or a quadrupole device.

In another embodiment, a counter-current device or a quadrupole device may be used on the laboratory benchtop. In further embodiments, a counter-current device or a quadrupole device may be used in sterile environments, for example a tissue culture hood, to maintain sterility of cell culture lines and samples. In certain embodiments, a counter-current device or a quadrupole device may be used in place of centrifugation to passage transformed, immortalized mammalian cell lines.

In other embodiments, the non-magnetic particles may be polymer beads. Polymer beads include, but are not limited to, synthetic polymers, natural polymers, synthetically modified natural polymers, and biodegradable polymers. In further embodiments, polymer beads may be separated from magnetic fluid using a counter-current or quadrupole device. In a further embodiment, polymer beads to be separated from magnetic fluid using a device of the present invention may be embedded with protein or small molecule compounds.

In certain embodiments, non-magnetic particles are separated from magnetic fluid using a counter-current or a quadrupole device wherein the separation of non-magnetic particles is based on the size of the particle. In further embodiments, gravity is not a significant force in the separation of non-magnetic particles from magnetic fluid using a counter-current or quadrupole device. In further embodiments, separation of non-magnetic particles from magnetic fluids using a counter-current or a quadrupole device is not based on differences in the density of the particles.

### Exemplification

The invention now being generally described, it will be more readily understood by reference to the following examples, which are included merely for purposes of illustration of certain aspects and embodiments of the present invention, and are not intended to limit the invention.

### EXAMPLE 1

### Magnetic Particle Synthesis

The magnetic fluid that the non-magnetic particles are mixed with is composed of magnetite particles (Fe₃O₄) coated with a graft copolymer layer that stabilizes the particle in water. The polymer coating used for the magnetic fluid is a graft copolymer of PAA-PEO/PPO. The PEO/PPO block copolymer grafted onto the PAA is the Jeffamine-2070 polymer by Huntsman, which contains PEO/PPO in a ratio of 70/30. The Jeffamine-2070 polymer is grafted onto the PAA by mixing the Jeffamine and PAA together in a flask and heating to approximately 180° C. for 2 hours while constantly bubbling nitrogen through the polymer.

The magnetic particles are typically about 32 nm in diameter, with the magnetite core itself being approximately 8-10 nm in diameter. Due to the small size of the magnetic particles, the water/magnetic particle mixture can be treated as a continuum, particularly when used for the separation of micron-sized non-magnetic particles, e.g. cells.

### EXAMPLE 2

### Experiments with the Counter-Current Flow Device

Experiments using both polystyrene (PS) beads and E. coli cells were performed with a counter-current device. The procedure for performing the experiments was the same in both cases. First, magnetic fluid of a specified concentration was mixed either with a solution of PS and water or with raw fermentation broth containing freshly grown E. coli cells. Only deionized water was used in all of the experiments. This mixture was then used as the feed and the total volume of feed for all experiments was 5 mL.

Next, the collection tube was filled with pure magnetic fluid of the same concentration as the magnetic fluid in the feed and clamped shut at the far left end. The presence of the magnetic fluid in the collection tube enhances the capture and sequestration of the non-magnetic particles that are carried into the tube by the movement of the magnets.

Once the collection tube was filled and clamped, all of the tubing, together with the T-junction, was securely placed in the device between the pairs of magnets. A 5 mL syringe was then filled with the feed, attached to a short inlet tube that led directly to the T-junction in the device, and placed in the syringe pump. The syringe pump was then used to pump the feed through the device at a specified flow rate (between 1.8 and 9.2 mL/hr). The clarified fluid that exited the device was then collected using a glass sample vial. Once the syringe was empty and all the feed had been pumped into the device, the syringe pump was turned off. However, the magnets were left to rotate for another 15 minutes to enhance the collection of non-magnetic particles from the 1 mL of feed fluid still entrained in the device.

Once the magnets were turned off, all of the tubing was removed from the device, and the fluid still entrained in the flow tube was collected, along with the fluid and non-magnetic particles contained in the collection tube. The contents of the collection tube, the fluid entrained in the flow tube, and the clarified fluid that exited the device were then analyzed. The analysis procedure was similar for both PS and cells and is summarized below.

Experiments with Polystyrene Beads

Experiments using the counter-current system were first performed using polystyrene (PS) beads as a model system. The beads, obtained from Spherotech, Inc. (Libertyville, Ill.), were spherical in shape with a diameter of 2.01±0.05 μm. The beads arrived as a suspension of 5.0 wt %±0.2 wt % beads in deionized water with 0.02% sodium azide added as bacteriostatic, and were used as received. The beads were negatively charged due to the presence of sulfate groups on their surface, and zeta potential measurements in 0.01 M NaCl (ionic strength 0.01 M) at a pH of 6.7 yielded an average zeta potential value of −104±3 mV.

Analytical Measurements

The concentration of polystyrene beads in the samples collected during the experiments was determined by measuring the absorbance (i.e. the optical density, or OD) of the samples at 600 nm using a Hewlett Packard UV-Visible Spectrophotometer (Model 8463). Since the optical density measurements at 600 nm can be correlated with the optical density for known polystyrene concentrations (the correlation has been shown to be linear), the concentration of the polystyrene beads in the sample fluids can be calculated using this correlation.

The magnetic nanoparticles in magnetic fluid also show absorbance at 600 nm, in addition to the absorbance observed from the polystyrene beads, and the two spectra are additive. The polystyrene concentration in the samples collected during experiments was therefore determined by first performing an initial measurement at 600 nm on a diluted solution of the experimental sample. A typical dilution was 0.05 mL sample in 10 mL deionized water. This measured absorbance provided the OD₆₀₀ value for the total mixture of both polystyrene and magnetic fluid. The diluted solution used for this initial measurement was then centrifuged at 4000 rpm for 40 minutes. This procedure sedimented only the polystyrene, since the magnetic nanoparticles that make up magnetic fluid are not affected significantly by centrifugation. The absorbance of the magnetic fluid supernatant at 600 nm was measured, and the difference between the total optical density of the full mixture and the optical density of just the magnetic fluid supernatant yielded the optical density of the polystyrene alone. Once the optical density was known for just the polystyrene, the polystyrene concentration was calculated using the previously determined OD₆₀₀ correlation.

Control Experiments

A control experiment was first performed with a counter-current device in which the feed consisted of 5 mL of 1 wt % PS in water, with no magnetic fluid added. This experiment was performed to determine if there were any unanticipated flow patterns in the device that would cause non-magnetic particles to enter the collection tube in the absence of magnetic fluid.

The collection tube was first filled with water, the tubing was assembled into the device, and then 5 mL of 1 wt % PS was pumped through the device at a flow rate of 2.8 mL/hr. The samples collected at the end of the experiment were then analyzed, and the results showed that less than 1% of the PS from the feed entered the collection tube. This result was exactly what was expected, with no separation of PS observed.

Magnetic Fluid Experiments

Experiments were then performed using magnetic fluid in the feed mixture to test the separation capability of a counter-current device. A feed mixture of 5 mL of 1 wt % PS and 1 wt % MF was used.

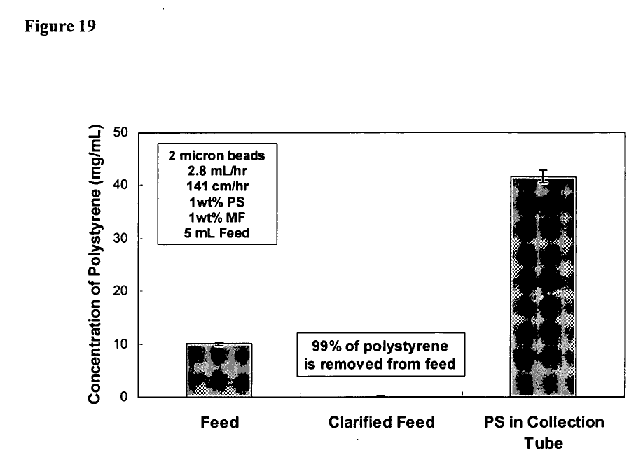

The collection tube was first filled with 1 wt % MF, the tubing was assembled into the device, and then 5 mL of 1 wt % PS and 1 wt % MF was pumped through the device at a flow rate of 2.8 mL/hr. The samples collected at the end of the experiment were then analyzed, and the results of the experiment are shown in FIG. 19.

The results show that excellent separation is achieved with the clarified fluid outflow containing less than 1% of the PS originally present in the feed stream. The material balance for the system also closes to within 1%, including the fluid retained in the flow tube in the device (the amount of PS retained in the system (in the flow tube) for this experiment was calculated to be 14.5% of the PS originally present in the 5 mL of feed, and the volume of the retained fluid was 26% of the total feed volume). This indicates that the analytical techniques used to determine the concentration of the PS in the feed and fluid samples is valid. Most importantly, the results show that magnetophoretic separation is successful with this device. Subsequent experiments using polystyrene beads under the same experimental conditions proved to be highly reproducible, achieving 99.3%±0.2% removal of the polystyrene beads from the feed fluid.

Experiments with E.coli Cells

Experiments were then performed using E. coli cells (wild strain BL2 1) in raw fermentation broth to determine how well the counter-current device functioned for cell clarification. The cells were cylindrically shaped and measured approximately 2-2.5 μm long by 1-1.5 μm in diameter, as determined by microscopy.

Analytical Measurements

The concentration of cells in the samples collected during experiments was determined using UV-Vis spectrophotometry (Hewlett Packard UV-Visible Spectrophotometer Model 8463) as described previously for the polystyrene beads. The optical density at 600 nm was measured for both the raw experimental samples and the centrifuged samples, and the difference between the OD₆₀₀ values was used to determine the optical density of just the cells alone. A linear correlation relating optical density to cell concentration was used to determine the concentration of the cells in each sample as wt % cells on a dry cell basis.

Magnetic Fluid Experiments

No control experiments were performed with the cells suspended in pure fermentation broth, with no magnetic fluid added. The importance of the magnetic fluid for separating non-magnetic particles from the bulk fluid was demonstrated with the PS beads. Instead, experiments were performed immediately using magnetic fluid in the cell feed mixture to test the ability of a counter-current device to clarify cells from raw fermentation broth.

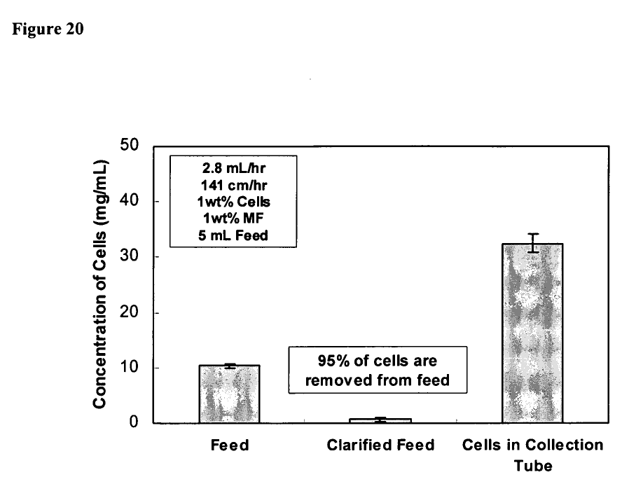

The results of a typical experiment are shown in FIG. 20, with a representative feed mixture composed of approximately 1.5 wt % cells and 1 wt % MF, and a feed flow rate of 1.8 mL/hr. The results show that magnetophoretic cell clarification is indeed possible with a counter-current device and is fairly successful in removing approximately 95% of the cells from the feed mixture with one pass through the system. The importance of the operating parameters (feed flow rate, concentration of MF and concentration of cells in the feed) was then tested and modeled with subsequent experiments.

Importance of the Operating Parameters

To test the importance of particular operating parameters on the separation capability of a counter-current device, a Box Behnken experimental design was followed. For the case of three parameters, the Box Behnken design assigns three values, or levels, to each of the three parameters in question. By performing experiments in various combinations of these levels, the effect of these parameters and the repeatability of the experiments can be determined.

In this case, the three operational parameters explored were fluid flow rate, magnetic fluid concentration, and cell concentration in the feed. The three values assigned to each parameter tested are shown in Table 1. The values for the cell concentration in the actual experiments varied over the range of 0.4 wt % to 1.9 wt %, but the values for the fluid flow rate and the concentration of MF in the feed were fixed in each experiment at one of the levels indicated in Table 1.

TABLE 1
Values of the Operational Parameters tested using a Box Behnken design
for a Counter-Current Device

| Fluid Flow | | |
|---|---|---|
| Rate (mL/hr) | wt % MF | wt % Cells |
| 1.8 | 0.5 | 0.5 |
| 5.3 | 1.0 | 1.0 |
| 9.2 | 1.5 | 1.5 |

The data gathered from using these values in experiments, as well as the data collected from earlier experiments, were then fit to an empirical model of the following form:
% Cells in Outflow=b₁+b₂x₁+b₃x₂+b₄x₃+b₅x¹²+b₆x₂²+b₈x₁x₂+b₉x₂x₃+b₁₀x₁x₃
where x₁ is the fluid flow rate, x₂ is the magnetic fluid concentration in weight fraction, x₃ is the cell concentration in weight fraction, and “% Cells in Outflow” refers to the mass of cells measured in the outflow divided by the total mass fed into the device, multiplied by 100.

The results of the empirical model show that the best separation (˜4.5% cells remaining) results from the lowest flow rate (1.8 mL/hr) at high magnetic fluid concentrations (<1.2 wt %), but also for relatively high cell content (0.7-1.1 wt %). Thus, cell concentration is not as important as flow rate and magnetic fluid concentration in obtaining good cell separation. In addition, the higher the magnetic fluid concentration, the faster the flow can be while still maintaining approximately the same degree of separation. The empirical model is not able to predict with better than 30% accuracy the specific percent of cell clarification that will be achieved in any one particular experiment given the precise fluid flow rate, magnetic fluid concentration, and cell concentration. It serves best as a good qualitative model, instead of a quantitative one. The results of all the experiments performed on a counter-current device that were used in the model are given in Table 2.

TABLE 2
Results from all the experiments performed with a counter-current device
used in the model to evaluate the importance of the fluid flow rate, the
concentration of MF, and the concentration of cells in the feed on the
separation capability of the device.

| Flow Rate | | | |
|---|---|---|---|
| (mL/hr) | wt % MF in Feed | wt % Cells in Feed | % Cells in Outflow |
| 9.2 | 1.0 | 0.48 | 7.1 |
| 1.8 | 1.0 | 0.52 | 11.3 |
| 1.8 | 1.0 | 1.44 | 6.5 |
| 9.2 | 1.0 | 0.65 | 5.1 |
| 9.2 | 1.0 | 1.88 | 20.1 |
| 9.2 | 1.5 | 0.93 | 9.8 |
| 9.2 | 0.5 | 1.10 | 31.0 |
| 1.8 | 1.5 | 1.12 | 4.3 |
| 1.8 | 0.5 | 0.91 | 7.4 |
| 5.3 | 1.5 | 1.42 | 15.9 |
| 5.3 | 1.5 | 0.43 | 3.9 |
| 5.3 | 0.5 | 0.51 | 26.4 |
| 5.3 | 0.5 | 1.71 | 37.3 |
| 5.3 | 1.0 | 0.73 | 14.3 |
| 5.3 | 1.0 | 0.68 | 13.0 |
| 5.3 | 1.0 | 0.68 | 13.0 |
| 2.8 | 1.0 | 1.47 | 11.9 |
| 2.8 | 1.0 | 1.13 | 7.2 |
| 2.8 | 1.0 | 1.38 | 14.8 |
| 2.8 | 1.0 | 1.02 | 5.4 |
| 2.8 | 1.0 | 1.01 | 2.0 |
| 9.2 | 1.0 | 0.88 | 14.0 |
| 9.2 | 1.0 | 1.16 | 20.2 |
| 9.2 | 1.0 | 0.98 | 15.0 |
| 1.8 | 1.0 | 0.50 | 10.9 |

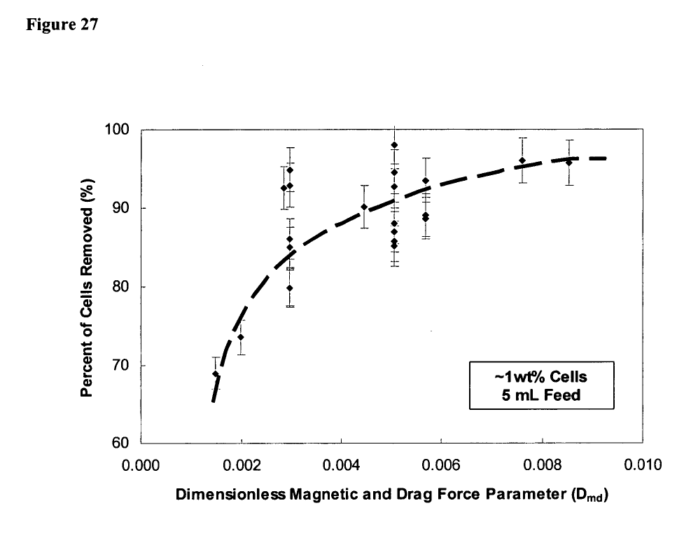

The dependence of the separation capability of the counter-current device on the dimensionless magnetic parameter, D_{md}, is shown graphically in FIG. 27, and depicts the percent of cells removed from the feed as a function of D_{md}, which was calculated from the values of the operating parameters used in each experiment. The percent of cells removed from the feed was calculated by dividing the mass of cells collected in the clarified feed fluid by the total mass of cells in the feed, then subtracting that ratio from 1, and multiplying by 100. The dashed line is present on the graph to indicate trends in the data and is not a theoretical prediction. The results show that better separation in the counter-current device was achieved for increasing D_{md}, as expected, since increasing D_{md} corresponds to an increase in the magnetic force on the cells.

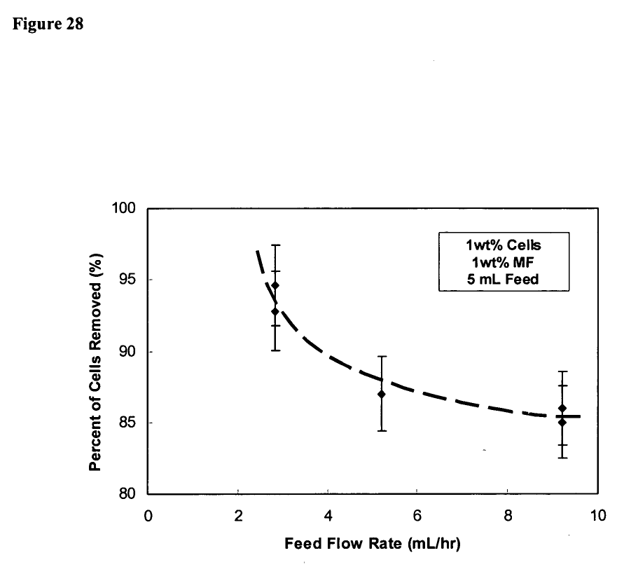

The percent of cells removed from the feed as a function of feed flow rate alone is shown in FIG. 28 for constant magnetic fluid and cell concentration, with the dashed line present on the graph to indicate trends in the data. It is evident that good cell separation is more easily achieved at lower flow rates than at higher flow rates, since higher flow rates translate into a stronger viscous drag force and a shorter residence time for the cells in the system, leading to less efficient capture of the cells by the traveling magnetic field.

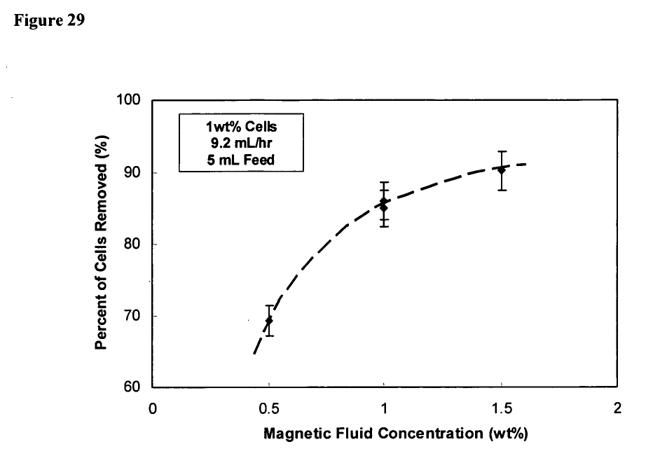

The percent of cells removed from the feed as a function of magnetic fluid concentration alone is shown in FIG. 29 for constant feed flow rate and cell concentration, with the dashed line present on the graph to indicate trends in the data. The flow rate used in these experiments was the highest flow rate used for all experiments with the counter-current device, 9.2 mL/hr. FIG. 29 shows that for constant feed flow rate and cell concentration, it is easier to achieve good cell separation with higher magnetic fluid concentrations than with lower magnetic fluid concentrations. In fact, the ability of the process to clarify cells from the feed drops dramatically with magnetic fluid concentrations less than 1 wt %. This result was anticipated, since it is the magnetization of the magnetic fluid that provides the magnetic force necessary for cell separation, and because magnetization scales linearly with the concentration of magnetic particles in the fluid, a lower concentration of magnetic fluid will result in a decreased magnetization of the fluid, and hence a smaller magnetic force on the cells. Thus, the higher the magnetic fluid concentration, the stronger the magnetic force on the cells and the better the separation, even at higher flow rates. The experimental data in FIG. 29 demonstrate this by asymptotically approaching 100% separation at higher magnetic fluid concentrations and rapidly approaching 0% separation (no separation) at lower magnetic fluid concentrations.

### EXAMPLE 3

### Model of a Quadrupole Device

One of the goals in designing a quadrupole device was to create a model of the device and test it virtually as well as experimentally. To this end, an equation was developed to determine the concentration profile of the cells in the feed as they traveled down the length of the cylinder in the device. The equation used takes into account fluid flow, diffusion, electrostatic effects between the particles, and magnetic force effects. The equation used is as follows, with the left hand side of the equation taking into account fluid flow, and the right hand side of the equation taking into account diffusion (first term), electrostatic effects (second term) and magnetic force effects on the cells (third term):

$$( {\tilde{v}}_{z} ( \tilde{r} ) - \tilde{g} ) \frac{\partial {\tilde{C}}_{p}}{\partial \tilde{z}} = \frac{1}{\tilde{r}} \frac{\partial }{\partial \tilde{r}} \tilde{r} ( \tilde{D} \frac{\partial {\tilde{C}}_{p}}{\partial \tilde{r}} + {\tilde{Ψ}}^{2} {\tilde{C}}_{p} \frac{\partial {\tilde{C}}_{p}}{\partial \tilde{r}} + \tilde{β} \tilde{M} ( \tilde{H} , \tilde{r} ) \frac{\partial \tilde{H} ( \tilde{r} )}{\partial \tilde{r}} {\tilde{C}}_{p} )$$

where the non-dimensional groups are defined as:

$$\begin{matrix}\tilde{D} = \frac{{W}_{f} {CD}_{p} L}{{v}_{max} {R}_{o}^{2} ρ} \\ \tilde{g} = \frac{{W}_{f} {CD}_{p} {\overline{V}}_{p} g}{{v}_{max} RT} ( 1 - \frac{{ρ}_{p}}{ρ} ) \\ {\tilde{Ψ}}^{2} = \frac{16 {π}^{2} {ɛψ}_{o}^{2} {R}_{p}^{2} {κ}^{- 2} ( 1 + 2 κ {R}_{p} ) {N}_{A}^{2} {C}_{po} {W}_{f} {CLD}_{p}}{RT ρ {R}_{o}^{2} {v}_{max}} \\ \tilde{β} = \frac{{μ}_{o} {\overline{V}}_{p} {M}_{s} {H}_{o} {W}_{f} {CLD}_{p}}{RT ρ {R}_{o}^{2} {v}_{max}}\end{matrix}$$

The equation describes the overall equation for magnetophoresis in the quadrupole design, and was used in this form in Matlab's PDE solver, PDEPE, to solve for the concentration profile of the non-magnetic particles as they moved through the column in the quadrupole device. Tabulated below are the values for all the constant parameters used for solving the equation.

TABLE
Values for the constant parameters used for solving the
overall equation for magnetophoresis for the quadrupole system.

| Constant Parameter | Value |
|---|---|
| μ₀ | 1.257 × 10⁻⁶ T · m/A |
| W_{f} | 0.02 kg/mol |
| C | 5.08 × 10⁴ mol/m³ |
| ρ | 1.017 × 10³ kg/m³ |
| R | 8.314 J/(mol · K) |
| T | 294 K |
| g | 9.80665 m/s² |
| ρₚ | 1.05 × 10³ kg/m³ |
| ε | 6.95 × 10⁻¹⁰ A · s/(V · m) |
| ψₒ | 0.083 V |
| κ⁻¹ | 9.61 × 10⁻⁷ m |
| Mₛ | 614 A/m |
| Hₜ | 43500 A/m |
| Hₒ | 499905 A/m |
| η | 0.995 × 10⁻³ kg/(m · s) |
| Rₒ | 0.01 m |
| L | 0.18 m |

Model Results for the Quadrupole Design

The equation was solved using Matlab's PDE solver, PDEPE, subject to the following initial and boundary conditions:

$$\begin{matrix}{\tilde{C}}_{p} = 1 for \tilde{z} = 0 ( all r ) \\ \frac{\partial {\tilde{C}}_{p}}{\partial \tilde{r}} = 0 for \tilde{r} = 0 , \\ \tilde{r} = 1 ( all z )\end{matrix}$$

where z=0 refers to the feed inlet portion of the column. Thus, the axial boundary condition sets the concentration at the column inlet equal to the initial non-magnetic particle concentration for all r, and the radial boundary conditions set the concentration flux equal to zero both at the centerline of the column and at the column walls for all z.

The Matlab model for the quadrupole design was initially constructed for use as a diagnostic tool to test slightly different geometries of the quadrupole design. Although most of the overall geometry of the quadrupole device was predetermined by outside factors, such as the overall length and outer diameter of the column, the geometry of the interior of the column had not been set. The equation was solved in Matlab to determine how the concentration profile of the non-magnetic particles was predicted to develop down the length of the column in order to determine when the concentration profile was fully developed (how far down the column) for a particle linear velocity. Based on the size and shape of the concentration profile, the dimensions of the inner cylinder that was designed to run coaxially inside the column at the outlet end (to remove the concentrated non-magnetic particles from the centerline of the column) could then be determined so the maximum amount of non-magnetic particles could be removed while minimizing the amount of bulk fluid removed from the system.

The predetermined dimensions of the quadrupole design were a column diameter of 2 cm and a column length of 20 cm, of which 18 cm was positioned directly within the four permanent magnets, which were each 18 cm long. The extra 2 cm of column length was used on the inlet side of the column to ensure that the velocity profile of the feed fluid was fully developed before entering the magnetic portion of the device. For the Matlab model, the point of zero column length (z=0) corresponds to the entrance into the magnetic field, which occurs 2 cm above the actual inlet of the column. Using this coordinate system, the outlet end of the column corresponds to 18 cm above the entrance to the field (z=18).

The results of the Matlab model for a feed fluid composed of 1 wt % non-magnetic particles in 1 wt % magnetic fluid with a maximum linear velocity along the centerline of 31.8 cm/hr, corresponding to a flow rate of 50 mL/hr, are shown in the Figure below, using 2 μm polystyrene beads as the non-magnetic particles for the system.

[image]

The concentration profiles for the polystyrene beads are nearly fully developed by the time the feed reaches 16 cm up the length of the column, and the results of this simulation were used establish the inner coaxial cylinder dimensions. Since the concentration profile was predicted to be fully developed by 16 cm, the inner coaxial cylinder was designed to extend 2 cm into the column from the outlet end. This allowed the concentrated non-magnetic particles at the center of the column to be removed from the bulk fluid at a point where their concentration was at a maximum (fully defined profile), but before the fluid hit the top wall of the cylinder, where the fluid flow profiles would be distorted. Thus, the extension of the inner coaxial cylinder into the column served to remove the concentrated non-magnetic particles while they were at their most concentrated with the minimum disruption of the flow pattern.

The Matlab model predicted that approximately 90% of the polystyrene beads would be concentrated in a area roughly 0.5 cm in diameter through the centerline of the column, where the peak of the concentration profile formed. Thus, an inner coaxial cylinder with a diameter of 0.5 cm was predicted to remove approximately 90% of the polystyrene beads from the feed while removing only 7% of the total volume of the bulk feed fluid.

The geometry of the flow column positioned inside the four magnets for the quadrupole design was therefore fully determined. The length of the column inside the magnets was 18 cm, with an extra 2 cm at the inlet to allow the velocity profile to be fully developed before the feed fluid entered the magnetic portion of the system. The overall length of the column was 20 cm, with a diameter of 2 cm. The inner coaxial cylinder at the outlet end of the column was 0.5 cm in diameter and extended 2 cm into the interior of the column to remove the maximum amount of concentrated non-magnetic particles from the centerline of the column while removing only 7% of the bulk fluid. This was the final column design that was used for experimental testing.

### EXAMPLE 4

### Experiments with a Quadrupole Device

Experiments using both polystyrene (PS) beads and E. coli cells were performed with a quadrupole device. The experimental procedure used for the quadrupole system was the same for all experiments performed. Two batches of fluid were prepared, the feed fluid itself (with a typical feed fluid volume of 160 mL) and the initial column fluid (typically 80 mL, the entrained volume of the device). The initial column fluid contained the same concentration of magnetic fluid as the feed but without the non-magnetic particles. The pH of each fluid was measured and recorded. The aluminum column and all tubing in the device were then quickly filled by hand with the initial column fluid, using a syringe attached to the inlet feed tube.

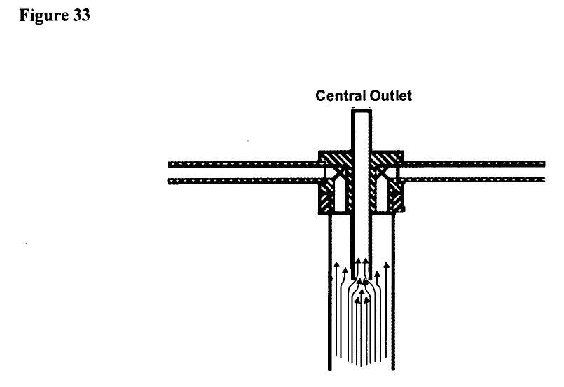

Once the entire system was full of magnetic fluid of the same concentration as the feed, the syringe was removed from the inlet feed tube, and the tube was place in the beaker containing the feed fluid. The peristaltic pump was turned on at a specified feed flow rate, and the needle valves attached to the outlets were adjusted so that 20% of the feed fluid exited from the central outlet and the remaining 80% of the feed exited through the two side outlets, 40% through each outlet. Although the coaxial inner tube that corresponds to the central outlet had a cross sectional area that was only 7% of the total cross sectional area of the column, a removal rate of 20% of the volumetric flow rate of the feed was selected to ensure that all concentrated non-magnetic particles would be removed, as an initial check of the separation capability of the quadrupole system. The flow patterns associated with this choice of flow rate for the central outlet are shown schematically in FIG. 33. The feed was also pumped through the device against the flow of gravity in all cases, with the feed inlet always positioned on the bottom and the outlets always positioned at the top. This configuration allowed for more efficient removal of the non-magnetic particles than if the feed were pumped in the same direction as the gravitational force.

The feed fluid in the beaker was stirred at regular intervals to prevent settling of the non-magnetic particles during the experiments. The fluid exiting the quadrupole device through each outlet was collected in glass sample vials and analyzed using UV-Vis spectrophotometry to determine the concentration of the non-magnetic particles in the outlet streams. At the end of the experiments, the aluminum column was removed from the magnet assembly and drained, and UV-Vis spectrophotometry was used to determine the concentration of the non-magnetic particles retained in the device.

Experiments with Polystyrene Beads

Experiments using the quadrupole system were first performed using polystyrene (PS) beads as a model system. The beads, obtained from Spherotech, Inc. (Libertyville, Ill.), were spherical in shape with diameters of 2.01±0.05 μm and 1.17±0.029 μm. The beads each arrived as a suspension of 5.0 wt %±0.2 wt % beads in deionized water with 0.02% sodium azide added as bacteriostatic, and were used as received. The beads were negatively charged due to the presence of sulfate groups on their surface, and zeta potential measurements in 0.01 M NaCl (ionic strength 0.01 M) at a pH of 6.7 yielded an average zeta potential value of −104±3 mV.

Analytical Measurements

The concentration of polystyrene beads in the samples collected during the experiments was determined by measuring the absorbance (i.e. the optical density, or OD) of the samples at 600 nm using a Hewlett Packard UV-Visible Spectrophotometer (Model 8463). Since the optical density measurements at 600 nm can be correlated with the optical density for known polystyrene concentrations (the correlation has been shown to be linear), the concentration of the polystyrene beads in the sample fluids can be calculated using this correlation.

The magnetic nanoparticles in magnetic fluid also show absorbance at 600 nm, in addition to the absorbance observed from the polystyrene beads, and the two spectra are additive. The polystyrene concentration in the samples collected during experiments was therefore determined by first performing an initial measurement at 600 nm on a diluted solution of the experimental sample. A typical dilution was 0.05 mL sample in 10 mL deionized water. This measured absorbance provided the OD₆₀₀ value for the total mixture of both polystyrene and magnetic fluid. The diluted solution used for this initial measurement was then centrifuged at 4000 rpm for 40 minutes. This procedure sedimented only the polystyrene, since the magnetic nanoparticles that make up magnetic fluid are not affected significantly by centrifugation. The absorbance of the magnetic fluid supernatant at 600 nm was measured, and the difference between the total optical density of the full mixture and the optical density of just the magnetic fluid supernatant yielded the optical density of the polystyrene alone. Once the optical density was known for just the polystyrene, the polystyrene concentration was calculated using the previously determined OD₆₀₀ correlation.

Control Experiments

The quadrupole process was first tested for its flow and magnetic properties to ensure that the physical system itself behaved as expected and introduced no anomalies during the separation process. Experiments were therefore performed using non-magnetic particles in the absence of magnetic fluid, to test the flow properties of the system alone, and magnetic fluid in the absence of non-magnetic particles, to test the magnetic properties of the system alone.

Control Experiment 1—Polystyrene in Water

A control experiment was performed with the quadrupole system in which the feed consisted of 160 mL of 1 wt % polystyrene in water, with no magnetic fluid added. This experiment was performed to determine if the polystyrene would be evenly distributed throughout the quadrupole system in the absence of the magnetic force caused by the presence of the magnetic nanoparticles. The size of the polystyrene beads used was 2.0 μm.

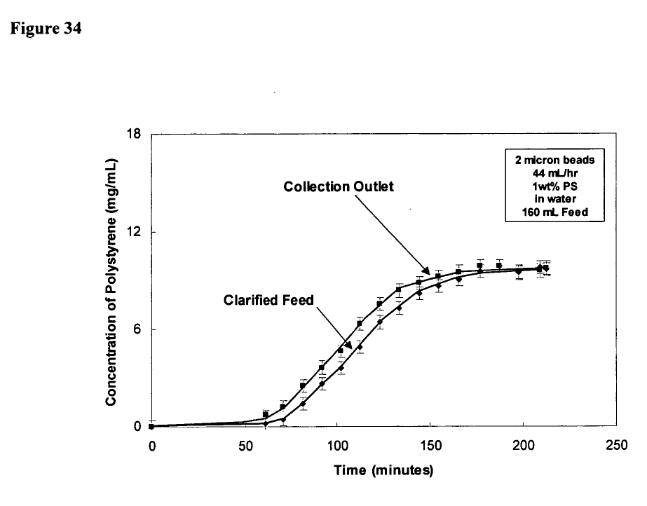

The quadrupole column and all tubing were filled with deionized water, after which 160 mL of 1 wt % polystyrene were pumped through the column at a flow rate of 44 mL/hr. The effluent exiting the top of the system from the three outlet streams was collected and analyzed using UV-Vis spectrophotometry to determine the concentration of polystyrene in the outlet streams. The results are shown in FIG. 34, and the concentration of polystyrene was found to be approximately the same in each outlet for the control experiment. At steady state, the inlet feed concentration was 9.8±0.4 mg PS/mL, the central collection outlet concentration was 9.8±0.4 mg PS/mL, and the two side outlets had an average concentration of 9.7±0.4 mg PS/mL. This was exactly the result that was expected, as no increase in concentration of the polystyrene beads was observed at the center of the column. There was also very little retention of polystyrene in the column during the experiment, with less than 3% of the polystyrene in the feed being retained in the device at the conclusion of the experiment. The overall material balance for the system also closed to within 5%, indicating that the analytical techniques used to determine the concentration of the polystyrene in the feed and fluid samples were accurate.

FIG. 34 shows that breakthrough of the polystyrene occurred at roughly 55 minutes and steady state concentration was achieved at approximately 160 minutes, as anticipated for a feed flow rate of approximately 44 mL/hr though a total device volume of 80 mL. Breakthrough of the polystyrene also occurred first through the central outlet (or collection outlet), which was also expected since the centerline of the column contains the fastest moving particles for a parabolic velocity profile. Thus, the quadrupole system was shown to function perfectly well on a physical flow level, and the analysis procedures for polystyrene content were also shown to be accurate.

Control Experiment 2—Magnetic Fluid Alone

Control experiments were conducted to test the magnetic properties of the system and determine how the strong magnetic field in the device affects the magnetic fluid itself, in the absence of non-magnetic particles.

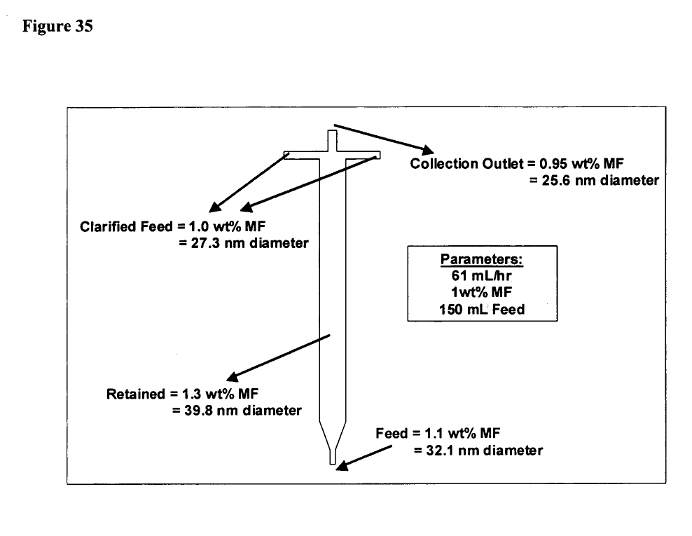

The quadrupole column and all tubing were filled with 1 wt % magnetic fluid (MF), after which 150 mL of 1 wt % magnetic fluid were pumped through the column at a flow rate of 61 mL/hr. The effluent exiting the top of the system from the three outlet streams was collected and analyzed using colorimetric iron analysis and dynamic light scattering (DLS) to determine the concentration of magnetite in the fluid and the size of the magnetic nanoparticles in the outlet streams. The results are given in FIG. 35, which shows that the concentration of magnetic fluid, defined as the concentration of magnetite in the fluid, increases in the column as the magnetic fluid feed is pumped through the device. For this experiment, 1.1 wt %±0.04 wt % MF entered the column, 0.95 wt %±0.04 wt % MF exited through the central outlet, and an average of 1.0 wt %±0.04 wt % MF exited through the two side outlets. Statistically, the outlet concentrations were all the same at approximately 1.0 wt %. These concentrations occurred immediately at the start of the experiment and remained steady for the duration. There was no breakthrough curve of any sort for the magnetic fluid concentration, indicating that the magnetic force inside the column immediately acted to retain approximately 10% of the magnetic nanoparticles as they entered the device.

Dynamic light scattering analysis showed that the particles retained in the column by the magnetic field were all the larger-sized nanoparticles. The magnetic fluid is composed of magnetic nanoparticles with an average hydrodynamic diameter of 31.6 nm±0.09 nm, but less than 1% of the particles on a number basis have a diameter greater than 60 nm. On a volume basis, and thus also on a weight basis, magnetic nanoparticles greater than 60 nm make up roughly 10% of the particles. The volume-average distribution differs from the number-average distribution in that each particle is weighted according to its size, with larger particles weighted more. This skews the average particle size towards higher values, but it also helps to uncover the presence of larger magnetic particles that exist in such low concentrations as to be inconsequential in the number-average distribution. It is these larger particles that are being retained in the column in the quadrupole system, as demonstrated by the statistically higher particle size of the retained fluid (39.8 nm±2.3 nm versus 32.1 nm±0.8 nm in the feed) and the statistically lower nanoparticle size of the fluid exiting the device (27.3 nm±1.8 nm in the two side outlets and 25.6 nm±0.8 nm in the central outlet versus 32.1 nm±0.8 nm in the feed).

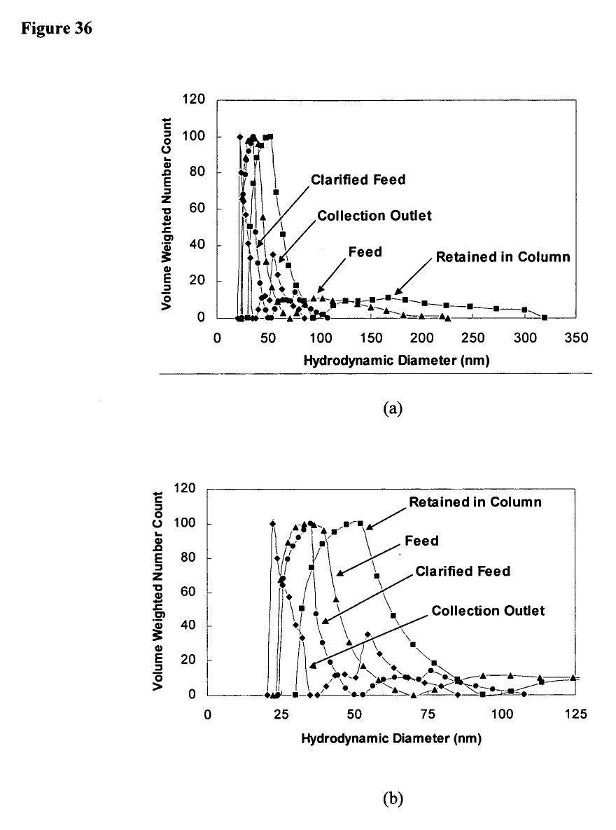

The retention of the larger particles in the column can be seen more clearly by looking at the volume-average distribution of magnetic particle size. The volume-average distributions for the feed, the outlets, and the fluid retained in the column at the end of the experiment are given in FIG. 36, which clearly shows how the smaller particles are eluted and the larger ones are retained in the column.

For the feed itself, roughly 10% of the particles by volume are larger than 60 nm, and approximately 6% are larger than 100 nm. For the clarified feed outlet (the two side outlets), roughly 8% of the particles by volume are larger than 60 nm, while only 2% are larger than 100 nm. Similarly, for the collection outlet (the central outlet), roughly 4% of the particles by volume are larger than 60 nm, and there are no particles present greater than 80 nm. However, for the retained fluid, approximately 20% of the particles are larger than 60 nm, with 11% by volume larger than 100 nm. This clearly shows that the larger magnetic particles in the feed fluid are being retained in the column. The retention of 20 vol % of particles larger than 60 nm is therefore expected, since the amount of feed fluid passed through the column was twice the working volume of the system, so twice the concentration of large particles in the feed should be retained.

The larger particles are retained because they represent aggregates of two or more single particles (those less than 60 nm in diameter) and therefore have a larger combined size for the magnetite core than do the single particles. This larger effective core size results in a stronger magnetic force attracting these particles to areas of high magnetic field in the system. Thus, the larger nanoparticles move towards the outer walls of the column towards the magnets, where they are retained by the strong magnetic field, while the smaller magnetic particles are left at the center of the column where the magnetic field is weakest.

The results of this control experiment demonstrate that the strength of the permanent magnets in the quadrupole device is sufficient to retain approximately 10 vol % of the magnetic particles that enter the device. This results in a higher concentration of magnetic fluid in the column, particularly near the column walls next to the magnets. This in turn helps to more efficiently push any non-magnetic particles in the column away from the walls, slightly enhancing the separation capability of the quadrupole process.

Polystyrene and Magnetic Fluid Experiments

Experiments using feed mixtures of magnetic fluid and non-magnetic 2 μm polystyrene beads showed that, in general, the quadrupole system operated according to expectations. Depending on flow conditions, the outlet streams exiting the device were of significantly different concentrations, with one of the streams being essentially clarified and the other containing a concentrated amount of the polystyrene particles.

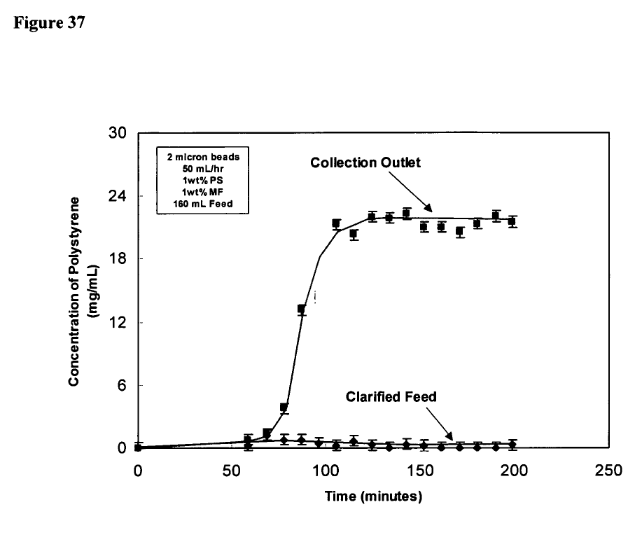

The quadrupole column and all tubing were filled with 1 wt % magnetic fluid, after which 160 mL of 1 wt % polystyrene (2 μm) and 1 wt % magnetic fluid were pumped through the column at a flow rate of 50 mL/hr. The effluent exiting the top of the system from the three outlet streams was collected and analyzed using UV-Vis spectrophotometry to determine the concentration of polystyrene in each outlet stream. The results are given in FIG. 37, which shows that the polystyrene was effectively removed from the feed through the central outlet. The overall material balance for the system closed to within 5% for this experiment.

The inlet feed concentration for the experiment was 9.6±0.4 mg PS/mL, the central collection outlet concentration was 21.5±0.9 mg PS/mL, and the two side outlets had an average concentration of 0.3±0.1 mg PS/mL. Additionally, at a flow rate of 50 mL/hr, the average residence time of the polystyrene in the system should be approximately 100 minutes, with initial breakthrough expected at 50 minutes for a parabolic velocity profile (Re<<1 for the device). This is essentially the behavior shown in FIG. 37, and in this case the polystyrene exited the column fairly sharply, indicating a buildup in polystyrene concentration as the beads traveled through the column. However, although the collection stream exiting the column was concentrated in polystyrene and the other effluent stream was essentially completely clarified, only approximately 40 percent of the polystyrene particles fed to the device were recovered in the outlets. The remainder of the polystyrene was retained in the system.

The significant retention of the polystyrene beads in the column was not the result of settling due to gravitational, or buoyancy, forces on the polystyrene beads. The theoretical settling velocity of the 2 μm beads, given by the terminal velocity of the beads as they settle in the magnetic fluid, was calculated using:

$${v}_{settling} = \frac{2 {R}_{p}^{2} g ( {ρ}_{part} - {ρ}_{fluid} )}{9 η}$$

where Rₚ is the radius of the beads, g is the gravitational constant, ρₚₐᵣₜ is the density of the beads, ρ_{fluid} is the density of the surrounding fluid, and η is the viscosity of the magnetic fluid and polystyrene mixture. For the 2 μm polystyrene beads, the theoretical settling velocity in magnetic fluid is 0.031 cm/hr, which is in good agreement with the experimentally determined settling velocity of 0.037±0.001 cm/hr, as measured by tracking the movement of the settling planes in columns filled with different concentrations of the polystyrene beads. This settling velocity is orders of magnitude smaller than the typical linear velocities used in this work, which were on the order of 20 cm/hr, and indicate that gravitational settling was not the cause of the retention of the beads within the device. Instead, the force acting on the polystyrene beads that causes the retention in the column appears to be magnetic in origin.

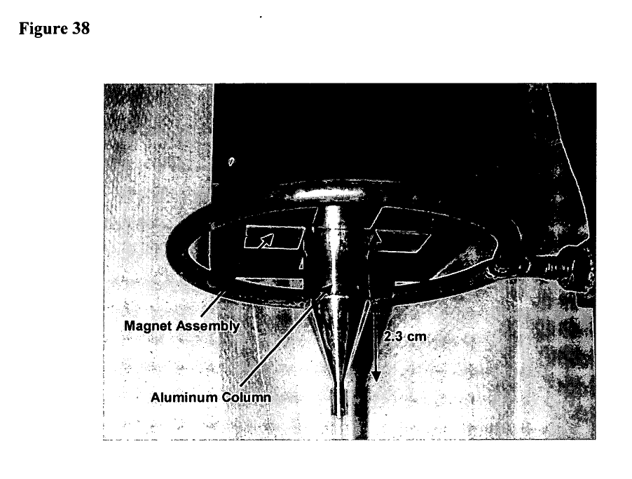

The same magnetic force that succeeds in separating the non-magnetic particles once they are in the device also acts as a barrier force against entry into the magnetic portion of the system. When the feed is first pumped into the column, it travels up through the cone at the inlet and then along 2 cm of the column length before reaching the magnet assembly, as shown in FIG. 38. Although the magnetic field inside the magnet assembly itself is radially symmetric and does not change along the length of the column, this is not true at the entrance to the assembly, where the magnetic field wraps around the long ends of the magnets. The resulting axial magnetic field gradients at the magnet edges cause end effects in which the downward magnetic force overcomes the upward drag force at certain radial positions in the column, which prevents the non-magnetic particles from entering the magnetized zones within the device, and so the axial magnetic field gradients provide a barrier to particle entry. The particles will migrate inwards towards the column centerline, however, owing to the radial components of the field gradient at these points within the column, and should eventually end up at a radial position where the drag force is sufficient to overcome the axial magnetic force, and the particle is able to enter the column. This upstream buildup of particles due to the axial magnetic gradient at the entrance to the magnet assembly in the device is most likely responsible for the retention of the particles observed within the column over the course of a run.

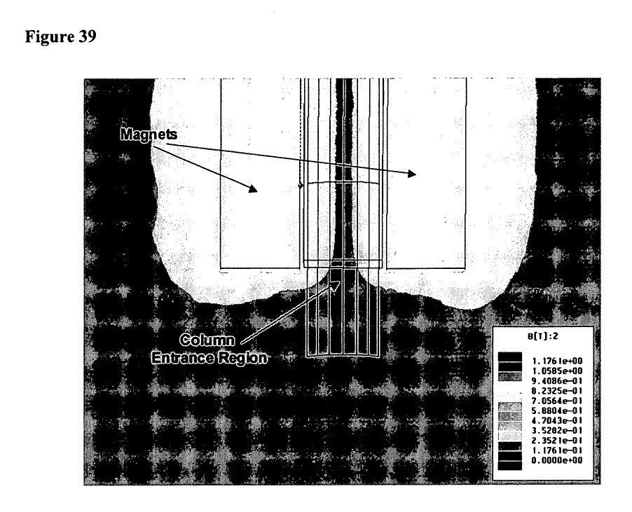

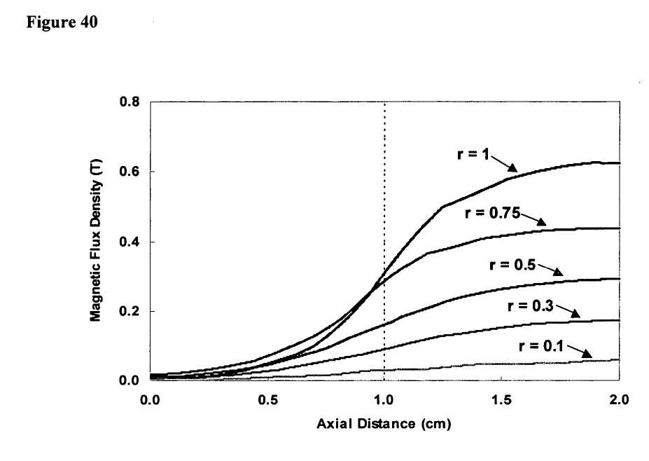

The magnetic barrier force can be quantified by modeling the axial and radial dependence of the magnetic field in the column at the entrance to the magnet assembly. Using the Maxwell 3-D Electromagnetic Field Simulator program (Ansoft Corporation), the magnetic flux density at the entrance to the magnet assembly was modeled for the quadrupole geometry. FIG. 39 shows a contour plot of the magnetic flux density established by the magnets at the entrance region into the magnet assembly, and the resultant axial field lines at the entrance are shown in FIG. 40. The simulation results, which were confirmed experimentally using a Gauss/Tesla meter, show a very sharp axial magnetic field gradient that occurs at the magnet edges and extends a quarter centimeter both into and out of the column, centered at the magnet edges. This sharp field gradient is strongest near the column walls and weakest at the column centerline, with the magnetic field degrading linearly along the radial axis. This axial magnetic field gradient is in addition to the expected radial magnetic field gradient established by the quadrupole orientation of the magnets.

The axial field lines were used to estimate the magnetic barrier force via the following equation:

$${F}_{m} = {μ}_{o} {V}_{p} M ∇ H where M = \frac{{M}_{s}}{{H}_{t} + H} H$$

where μ₀ is the permeability of free space, Vₚ is the volume of the non-magnetic particles, M is the magnetization of the magnetic fluid, ∇H is the magnetic field gradient, Mₛ is the saturation magnetization of the magnetic fluid at high magnetic field strengths, H is the magnetic field, and Hₜ is the magnetic field strength at which the magnetization of the magnetic fluid is half the saturation magnetization. The magnetic force is constant for constant non-magnetic particle size and constant magnetic fluid concentration.

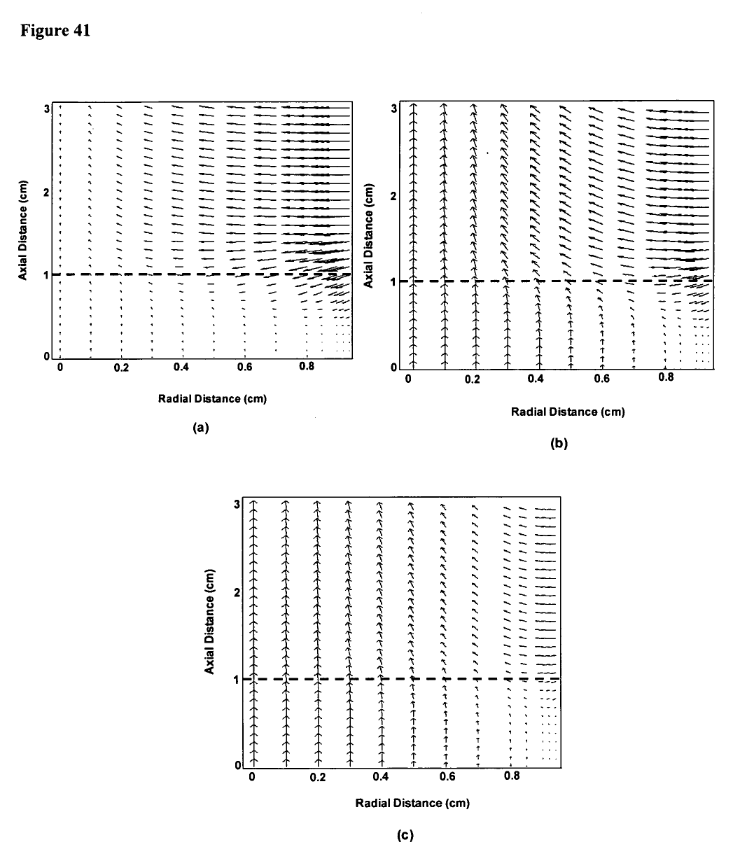

A force balance on the non-magnetic particles at various locations in the entrance region to the magnet assembly was used to determine the trajectories of the non-magnetic particles as they entered the column and flowed through the device. The magnetic forces in both the axial and radial directions were opposed by the drag force from the motion of the particles, with the drag force in the axial direction enhanced by the fluid flow up the column. FIG. 41 shows the magnitudes and directions of the net migration velocities the particles would experience if they were to be placed at different positions within the column. It is clear that at some locations within the column, the net migration of the particles is downward, and hence the particle motion would be reversed, particularly at low flow rates, with the particles near the column wall affected most because of the lower flow velocities and higher field gradients present there. These particles will accumulate at points just where the downward magnetic forces are balanced by the upward drag associated with the local flow within the column. Complete retention is not predicted, however, as there would still be small radial components of the magnetic force that ensure some radial migration of the particles to the faster flowing regions near the column centerline.

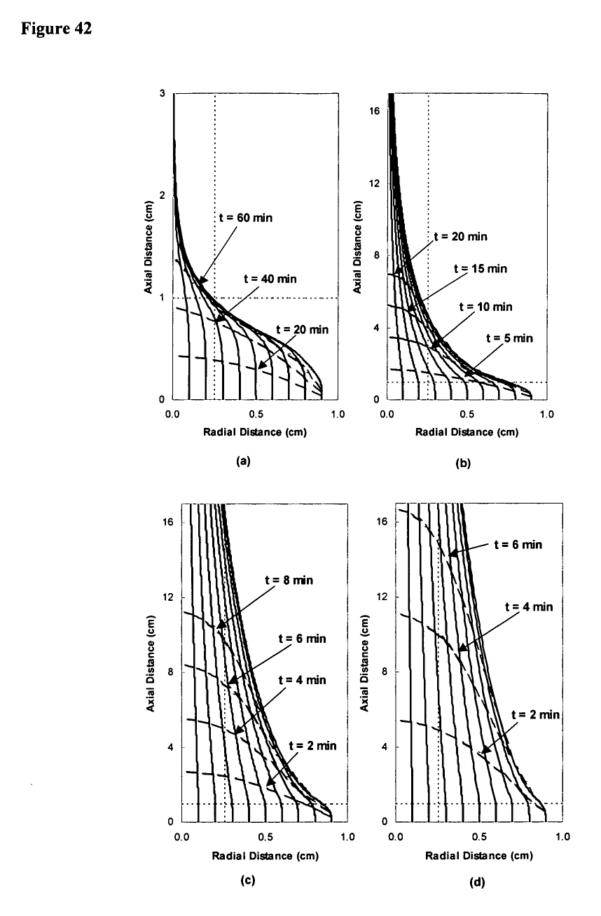

Particle trajectories for particles entering the column at different radial positions were determined by integrating the equation:

$$\underline{v} ( t ) = \frac{ⅆ \underline{r} ( t )}{ⅆ t} i . e . \underline{r} ( t ) = \{ r ( t ) , z ( t ) \} = \underline{r} + {\int }_{0}^{t} \{ {v}_{r} ( r , z ) , {v}_{z} ( r , z ) \} ⅆ t$$

where r(t) and z(t) are the radial and axial positions, respectively, for a particle at a time t after being introduced to the column at position rₒ at time t=0, and vᵣ and v_{z} are the components of the particle velocities at position {r(t),z(t)}, as shown in FIG. 41. The calculated trajectories for 2 μm non-magnetic particles are shown in FIG. 42 for different average flow rates through the column.

FIG. 42 clearly shows that the particles are deflected by the axial and radial magnetic forces at the entrance to the magnet assembly and become substantially concentrated in the first few centimeters of the column at lower flow rates, due to the relative dominance of the magnetic force over the drag force in this region. At higher flow rates, only the slowest moving particles at the column walls experience a significant enhancement in concentration, due to the overall dominance of the drag force at higher flow rates. The trajectories show that lower flow rates will result in better separation of the non-magnetic particles, and although the greatest extent of enhanced particle concentration in FIG. 42 occurs at flow rates significantly lower than those used in this work, the trajectories clearly show how the particles can become very concentrated at the centerline even for moderate flow rates. Clearly, at the low flow rates, it would also be best to draw off only a small amount of the fluid flowing through the central collection outlet in order to maximize the concentration in the collected fraction while minimizing the loss of the bulk fluid.

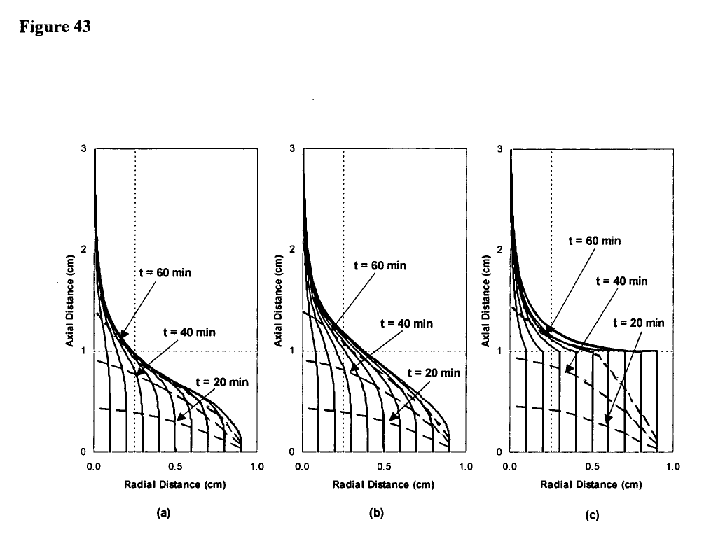

Also shown for comparison in FIG. 43 are the trajectories the particles would follow for low flow rates in the absence of the axial magnetic field gradient in the entrance region while still in the presence of the radial entrance gradient, as well as for the case of no radial or axial entrance magnetic field gradients. The trajectories clearly show the large effect on particle movement and concentration that the magnetic gradients have in the entrance region. For a flow rate of 2 mL/hr, a particle at the column wall will travel from 0 to 3 cm up the column (with the magnets present at 1 cm) in 515 minutes for the case that excludes both axial and radial entrance fields. For the case that includes only the radial entrance fields, a non-magnetic particle will take 276 minutes to travel the same distance, while for the case including the full axial and radial magnetic field gradients at the entrance, a particle will take 420 minutes to travel that distance. The particle trajectories represent single particles calculated in the absence of particle-particle interactions, and thus do not describe the full behavior of particle flow and retention in the column; however, even this single particle force balance clearly shows that the axial gradients present in the entrance region in the device do have a retarding effect on the motion of the particles, which increases their residence time in the column when compared to the case where only radial entrance gradients are present.

Experimental proof of the presence of the magnetic barrier force was provided by passing a feed fluid composed of 1 wt % 2 μm polystyrene beads in 1 wt % magnetic fluid through the system at 30 mL/hr in both the presence and absence of the magnetic field. The polystyrene beads were retained in the column when the magnets were in place, but without the magnets, all of the polystyrene exited the system, with a mass balance closure to within 2%. No polystyrene separation was achieved, as expected, but the negligible retention rate in the absence of the magnets showed that the accumulation of polystyrene in the magnetized column is the result of the balance of forces between the magnetic force at the entrance to the magnet assembly and the drag force exerted by the fluid flow.

Effects of Operating Parameters on Polystyrene Particle Separation and Concentration

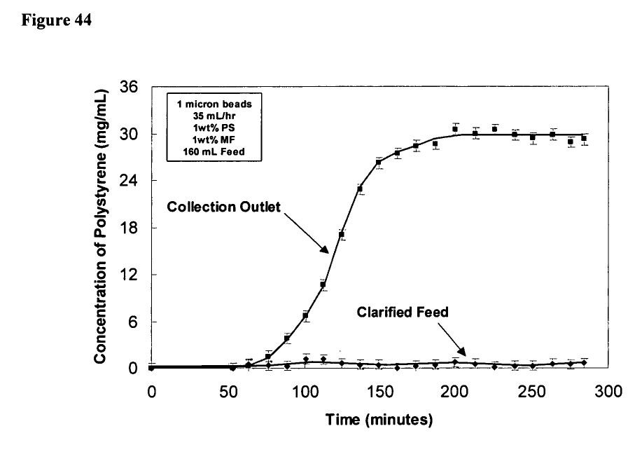

Fluid flow rate and polystyrene bead size were both varied in the quadrupole system to determine how differences in these operating parameters affected the separation capability and ideal operating range of the device. Polystyrene beads with a diameter of 1.17 μm were used to determine the effect of particle size on the separation capability of the quadrupole process. The quadrupole column and all tubing were filled with 1 wt % magnetic fluid, after which 160 mL of 1 wt % polystyrene (1 μm) and 1 wt % magnetic fluid were pumped through the column at a flow rate of 35 mL/hr. The effluent exiting the top of the system from the three outlet streams was collected and analyzed using UV-Vis spectrophotometry to determine the concentration of polystyrene in each outlet stream. The results are given in FIG. 44, which shows that the polystyrene was quite effectively removed from the feed through the central outlet. The overall material balance for the system also closed to within 5% for this experiment, indicating that the analytical techniques used to determine the concentration of the 1 μm polystyrene beads in the feed and fluid samples were accurate, even in the presence of magnetic fluid.

The inlet feed concentration for the experiment was 9.7±0.4 mg PS/mL, the central collection outlet concentration was 29±1 mg PS/mL, and the two side outlets had an average concentration of 0.6±0.1 mg PS/mL. This resulted in a removal of approximately 99% of the polystyrene when compared to the effluent from the two side outlet streams. Less polystyrene was retained in the column for this experiment than for the corresponding experiment with the 2 μm beads. The retention rate at 35 mL/hr for the 1 μm beads was approximately 30%, indicating that 70% of the feed that entered the column exited through the outlets. This decrease in retention was expected, since the change in particle size affects both the magnetic force and the drag force on the non-magnetic particles, with a larger effect on the magnetic force, which scales with the cube of the radius of the particles while the drag force scales simply with the radius. Decreasing the particle size should therefore result in less accumulation in the column for the 1 μm polystyrene beads compared to the 2 μm beads even at lower flow rates, since the axially directed magnetic force is weaker for smaller particles, leading to less accumulation of the polystyrene at the entrance to the magnet assembly, as was observed.

The weaker magnetic force on the 1 μm particles resulted not only in less accumulation of polystyrene in the column, but also in a less intense buildup in polystyrene concentration as the beads traveled through the column, resulting in a breakthrough curve that was less sharp when compared to the corresponding case for the 2 μm particles. At a flow rate of 35 mL/hr, the average residence time of the polystyrene in the system should be approximately 140 minutes, with initial breakthrough expected at 70 minutes for a parabolic velocity profile (Re<<1 for the device). This is exactly the behavior shown in FIG. 44, indicating that the axial magnetic force on the smaller particles is weak enough not to hinder their motion appreciably through the column, even though the force is still strong enough at this flow rate to retain roughly 30% of the particles.

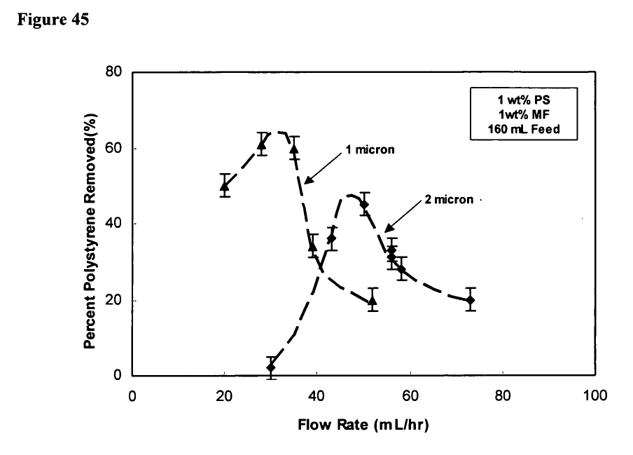

The effect of feed flow rate on the recovery and concentration of 1 and 2 μm polystyrene beads is shown in FIG. 45 for feeds containing 1 wt % polystyrene in 1 wt % magnetic fluid. The curves for the two sets of beads are similar in that the recovery of polystyrene in the collection outlet exhibited a maximum at some intermediate flow rate, and then declined as the flow rate increased. This decrease in recovery at higher flow rates was anticipated because faster flows translate into a decreased residence time for the polystyrene beads in the column, resulting in fewer beads that are able to migrate to the center of the column under the magnetic force before exiting the device. The results for the lower flow rates, however, were unexpected, as they showed poorer separation even though theoretically the residence times were sufficient for the particles to migrate to the center of the column before exiting the device. These effects were attributed to the accumulation of polystyrene in the column, and hence to the fact that the column had not attained steady state operation, even though the effluent concentrations were unchanging with time for the duration of the experiments. The percent removal of polystyrene beads from the collection outlet was calculated based the amount of polystyrene fed into the device. Thus, if a significant fraction of the polystyrene was retained in the column, the apparent separation capability of the process would decrease, as was observed.

The effect of particle size in the system is as expected, since the smaller particles are less responsive to the applied magnetic field gradients, and hence require longer residence times to effectively migrate to the column centerline for removal from the central outlet. Thus, the entire curve for the 1 μm particles is shifted to lower flow rates relative to the curve for the 2 μm particles. The peak removal efficiency for the 1 μm particles is higher than that for the larger particles, because the smaller particles experience less of a magnetic barrier force, and hence a lower retention rate, upon entering the magnetic portion of the column, and therefore more of the feed from the 1 μm particles is eluted in the outlet channels, even at lower flow rates.

These results for the 1 and 2 μm beads also indicate an interesting side-benefit of the retention of non-magnetic particles in the quadrupole system, which can be exploited for fractionation based on size. Since the ideal operational range for 1 μm particles lies below the ideal operational range for 2 μm particles, the quadrupole process could be used for the separation of particles of different sizes, where, for example, the system was operated at a flow rate low enough to retain all of the 2 μm sized particles in a mixture while eluting and concentrating the 1 μm particles, in this case utilizing a flow rate of approximately 30 mL/hr.

FIG. 45 shows the percent of polystyrene removed through the central collection outlet based on the incoming feed concentration. However, a better measure of the separation capability of the quadrupole process is the amount of polystyrene beads remaining in the clarified feed exiting the system from the two side outlets, since a low concentration of polystyrene in the side outlets represents excellent clarification of the feed, regardless of whether the polystyrene exits through the central outlet or is retained in the system. In addition, if the percent removal of polystyrene is recalculated based on the ratio of the polystyrene collected in the central outlet to the polystyrene collected in the side outlets, the results yield a pseudo steady state approximation of the separation capability of the device, since all of the polystyrene that makes it past the entrance to the magnetic assembly should no longer be affected by axial magnetic field gradients, and will be eluted at the top of the column.

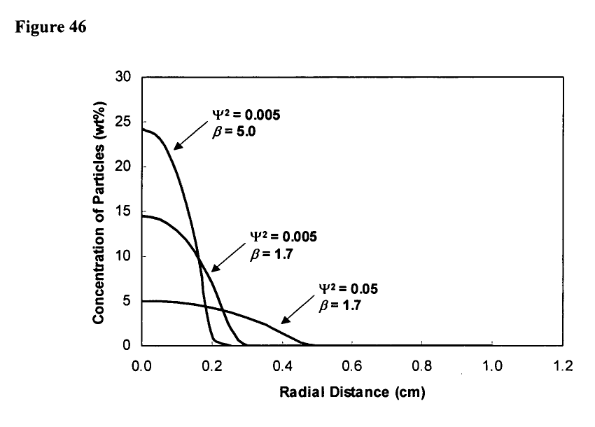

This pseudo steady state operation can be modeled. Neglecting diffusion and gravity, which were shown to be negligible, the governing equation for pseudo steady state operation of the quadrupole process is given by the following equation:

$$( 1 - {\tilde{r}}^{2} ) \frac{\partial {\tilde{C}}_{p}}{\partial \tilde{z}} = \frac{1}{\tilde{r}} \frac{\partial }{\partial \tilde{r}} \tilde{r} ( {\tilde{Ψ}}^{2} {\tilde{C}}_{p} \frac{\partial {\tilde{C}}_{p}}{\partial \tilde{r}} + \tilde{β} \tilde{M} \frac{\partial \tilde{H}}{\partial \tilde{r}} {\tilde{C}}_{p} )$$

where \tilde{Ψ}² and \tilde{β} are the dimensionless groups representing the electrostatic repulsive forces and the magnetic forces, respectively. The particle concentration profiles predicted by this model for different values of the parameters \tilde{Ψ}² and \tilde{β} for 2 μm non-magnetic particles are given in FIG. 46, which shows that both parameters play an important role in particle separation. An increase in the magnetic parameter results in an increase in the effectiveness of the separation of the particles by more strongly forcing them towards the centerline, while an increase in the electrostatic parameter results in a decrease in separation, since the electrostatic term represents a repulsive force between particles that acts to prevent their concentration at the centerline.

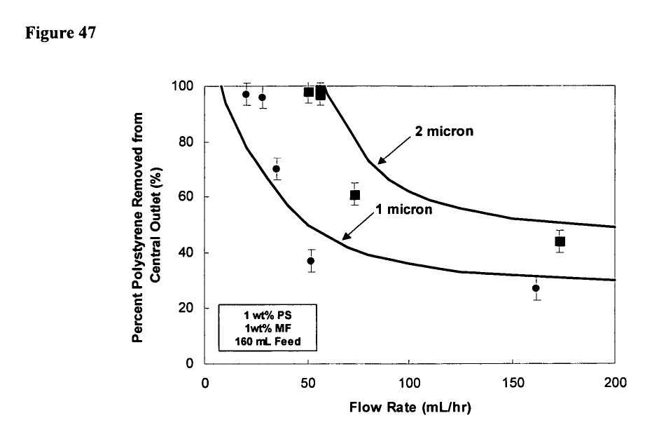

The model predictions of the concentration profiles were used to estimate the fraction of the polystyrene beads leaving the column through the central collection outlet. The model results are compared in FIG. 47 with the experimentally measured values of polystyrene separation, based on the ratio of the polystyrene collected in the central outlet to the polystyrene collected in the side outlets, where the electrostatic group in the model was used as an adjustable parameter to fit the experimental data. A value for the electrostatic group equal to approximately 0.05 provided the best balance between the magnetic and electrostatic forces and the best fit to the experimental data.

The fit of the model to the experimental results is good for both the 1 μm and 2 μm beads, and captures the general trend of excellent separation at low flow rates and poorer separation at higher flow rates, where the particles do not have a sufficiently long residence time to achieve good separation. For the 1 μm beads, the model predicts that at high flow rates, the percent recovery of the polystyrene beads should approach roughly 30%, while for the 2 μm beads, the percent recovery should approach roughly 45%, although both eventually asymptote to 20% at extremely high flow rates. Experimentally, the beads both approach 20% recovery at moderately high flow rates, since the central outlet is always operated at 20% of the feed flow rate. Even though the model overpredicts the percent recovery at these moderately high flow rates, it does adequately capture the separation capability of the process at lower flow rates. Indeed, at low flow rates for both particle sizes, separation as high as 99% was achieved experimentally, indicating that the device functions quite well as a clarification system, even with the initial retention of the particles at the magnet assembly entrance.

Experiments with E. coli Cells

The separation of E. coli cells (wild strain BL21) from raw fermentation broth was explored using the quadrupole process. The cells were cylindrically shaped and measured approximately 2-2.5 μm long by 1-1.5 μm in diameter, as determined by microscopy. The following sections discuss the results of the experiments performed using the cells in the quadrupole system.

Analytical Measurements

The concentration of cells in the samples collected during experiments was determined using UV-Vis spectrophotometry (Hewlett Packard UV-Visible Spectrophotometer Model 8463) as described previously for the polystyrene beads. The optical density at 600 nm was measured for both the raw experimental samples and the centrifuged samples, and the difference between the OD₆₀₀ values was used to determine the optical density of just the cells alone. A linear correlation relating optical density to cell concentration was used to determine the concentration of the cells in each sample as wt % cells on a dry cell basis.

Control Experiments

A control experiment was performed with the cells in which the feed consisted of 150 mL of 0.47 wt % cells on a dry cell basis in fermentation broth, with no magnetic fluid added, to determine if the cells would be evenly distributed throughout the quadrupole system in the absence of the magnetic force caused by the presence of the magnetic nanoparticles.

The quadrupole column and all tubing were filled with deionized water, after which 150 mL of 0.47 wt % cells in fermentation broth were pumped through the column at a flow rate of 56 mL/hr. The effluent exiting the top of the system from the three outlet streams was collected and analyzed using UV-Vis spectrophotometry to determine the concentration of the cells in the outlet streams. The inlet feed concentration was 4.7±0.2 mg cells/mL, the central collection outlet concentration was 3.8±0.2 mg cells/mL, and the two side outlets had an average concentration of 4.0±0.2 mg cells/mL. This was exactly the result that was expected, as no increase in concentration of the E. coli cells was observed at the center of the column. However, the decrease in the effluent concentrations when compared to the feed concentration shows that the cells did experience some settling in the device due to natural cell flocculation, which amounted to approximately 15% of the feed concentration of the cells, and indicates that the system never reached a truly steady state operation despite constant effluent concentrations during the course of the run.

The overall material balance for the system closed to within 5%, demonstrating that the analytical techniques used to determine the concentration of the cells in the feed and fluid samples were accurate. Thus, the quadrupole system was shown to function as expected on a physical level for E. coli cells, and the analysis procedures for the cell content in the experimental samples were shown to be accurate.

Cells and Magnetic Fluid Experiments

Experiments were performed using a feed mixture of both magnetic fluid and E. coli cells to test the full separation capability of the quadrupole system for magnetophoretic cell clarification. Experiments were performed using a constant feed composition of 1 wt % magnetic fluid and 0.5 wt % cells on a dry cell basis using feed flow rates ranging from 47 mL/hr to 67 mL/hr. The results of each of these experiments were identical, and showed that greater than 95% of the cells entering the device were retained in the column, resulting in no real magnetophoretic separation by the system.

Several experiments were performed to investigate the cause of the retention of the cells in the column. Since the cells did not show such a high degree of retention when used in the device without magnetic fluid, the cause of the retention was determined to be related to either the magnetic fluid itself or to the magnetic properties of the system. Experiments performed using the same feed concentrations of cells and magnetic fluid but without the presence of the magnets showed the same high level of retention of the cells. This result indicated that the cell retention was not entirely related to the forces exerted by the magnetized magnetic fluid, but that the magnetic fluid itself was inducing the cells to form aggregates, which were then settling in the column. Further experiments showed that the aggregates were not the result of any lysing of the cells, but were simply loose clumps of cells that were easily dispersed by mechanical agitation of the system. Thus, the magnetic fluid was flocculating the cells and inducing the formation of large cell aggregates.

The large cell aggregates were the cause of the settling behavior observed in the quadrupole device. Theoretically, individual E. coli cells should have a settling velocity in fermentation broth of around 0.05 cm/hr. Experimentally, the measured settling velocity of cells in fermentation broth was determined to be 0.07-0.3 cm/hr by tracking the movement of the settling plane in columns filled with different concentrations of the cell suspensions. The settling plane was not sharply defined for the cell suspensions, however, so the measured settling velocity is a rough approximation. When mixed with magnetic fluid, individual E. coli cells have a theoretical settling velocity of 0.04 cm/hr. Experimentally, however, cells mixed with magnetic fluid were shown to settle with a velocity of roughly 1.5 cm/hr at all cell concentrations tested, up to 1.1 wt % cells. Based on the experimentally measured settling velocity, the cells appear to aggregate into loose clumps roughly 12 μm in diameter (assuming spherical aggregates), with an estimated number of cells per aggregate of approximately 151. The settling velocity results would indicate that a linear flow velocity greater than 1.5 cm/hr would be sufficient to overcome the settling of the cell aggregates in the system. Experimentally, however, cells were still retained in the device in the absence of a magnetic field for an average linear velocity as high as 21 cm/hr (59 mL/hr), which would indicate an average cell aggregate size of 46 μm. The mechanism behind the formation of the aggregates in the presence of magnetic fluid is not fully understood, however, and so it is possible that differences in the operation and set up of the experiments could account for the discrepancy in the calculated aggregate sizes. In either case, it is clear that the interactions between the magnetic fluid and the cells result in the formation of cell aggregates in the quadrupole system, which are subsequently responsible for the high rate of accumulation of the cells in the device at the same flow rates used for the polystyrene experiments.

Preliminary testing at higher pH levels was used with the goal of increasing the negative surface charge on the magnetic nanoparticles and E. coli cells, thus increasing the electrostatic repulsive forces between them in a effort to reduce the amount of cell flocculation. However, although higher pH levels do correspond with slightly higher surface charge on the magnetic nanoparticles, testing showed that higher pH levels do not show a corresponding increase in the negative surface charge on the E. coli cells, and so increasing the working pH level would have little effect on preventing the flocculation of the cells through increased electrostatic repulsion.

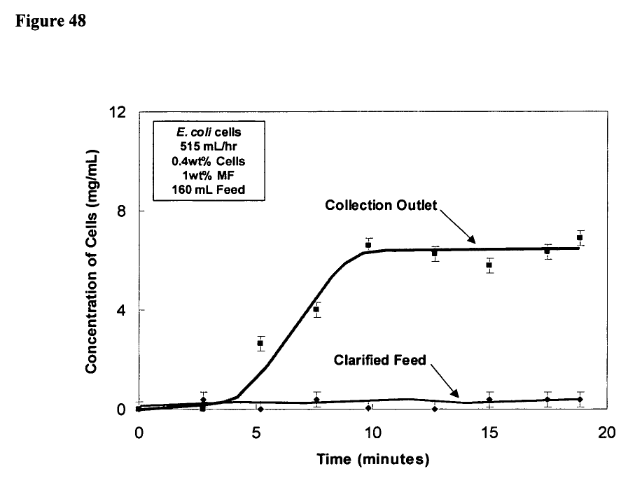

Experiments were performed instead to take advantage of the cell flocculation, utilizing much higher flow rates than were used for the polystyrene experiments, thus overcoming the settling of the cell aggregates by simply increasing the drag force on them. The quadrupole column and all tubing were filled with 1 wt % magnetic fluid, after which 160 mL of 0.4 wt % E. coli cells on a dry cell basis and 1 wt % magnetic fluid were pumped through the column at a flow rate of 515 mL/hr. The effluent exiting the top of the system from the three outlet streams was collected and analyzed using UV-Vis spectrophotometry to determine the concentration of the cells in each outlet stream. The results are given in FIG. 48, which shows that the cells were quite effectively removed from the feed through the central outlet at the high flow rate.

The inlet feed concentration for the experiment was 4.0±0.1 mg cells/mL, the central collection outlet concentration was 6.9±0.3 mg cells/mL, and the two side outlets had an average concentration of 0.4±0.1 mg cells/mL. Thus, roughly 95% of the cells were removed, compared to the concentration of cells in the side outlet streams. At a flow rate of 515 mL/hr, the average residence time of the cells in the system should be approximately 9 minutes, with initial breakthrough expected at 4 minutes for a parabolic velocity profile (Re<<1 for the device). This is essentially the behavior shown in FIG. 48, indicating that the axial magnetic force on the cell aggregates was not strong enough to hinder their motion appreciably through the column at the flow rate used in this experiment. Based on these results, calculations involving the balance between the magnetic forces and drag forces on the cells at this flow rate would indicate a cell aggregate size closer to 12 μm than to 46 μm for this experiment. It is probable that the average size of the cell aggregates is variable depending on the operating conditions of the device, since the mechanism of flocculation is not fully understood, and observations of the cell flocculates show that they are easily dispersed with mechanical agitation of the system.

Even at such a high flow rate, the cells still settled significantly in the column due to the high axial magnetic force on the large flocculated cell aggregates, with approximately 54% of the cells retained in the device. The presence of the cell aggregates also affected the flow through the central outlet valve, which became fouled during the experiment, thereby decreasing the actual amount of exiting cells. A sample of the fluid located directly upstream of the central outlet valve was collected at the conclusion of the experiment, and showed a cell concentration of 9.3±0.5 mg cells/mL. Thus, the quadrupole system was operating with an even better separation capability than the initial concentration profiles would imply, since some of the cells were unable to exit the device due to fouling of the central collection outlet valve, which was not originally designed to accommodate large particulate flows. Samples upstream of the side outlet valves showed no such increase in cell concentration.

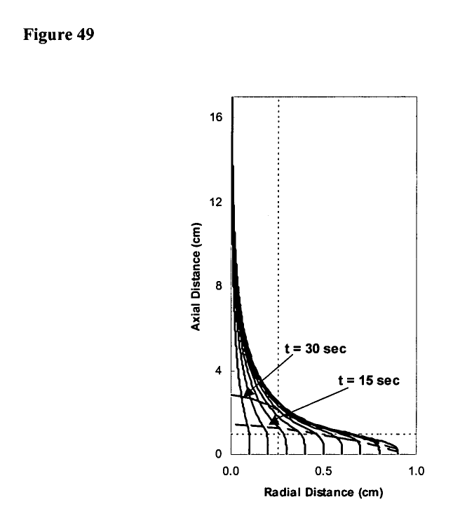

The trajectory of the cells in the quadrupole device was calculated for the case of 12 μm cell aggregates, and the results are shown in FIG. 49 for a flow rate of 500 mL/hr. The general shape of the trajectories are similar to the case for low flow rates with the 1 and 2 μm polystyrene beads, indicating that even though the flow rate is significantly higher for the cell experiments, the magnetic force pushing the cell aggregates to the centerline is enhanced due to the larger volume of the aggregates. Thus, good separation of the cells can be achieved in the quadrupole system even at high flow rates. A flow rate of 515 mL/hr was the highest flow rate tested using the E. coli cells. Flow rates lower than 515 mL/hr also showed similarly good separation efficiency, with over 95% of the cells removed when compared to the effluent in the side outlet streams. This is consistent with the pseudo steady state model predictions, which predict a 98% removal of cells for flow rates of 515 mL/hr or less, using an average cell aggregate size of 12 μm. However, lower flow rates also resulted in an increased retention of the cells in the device, with an experimental retention rate of 80% for a flow rate of approximately 360 mL/hr, further demonstrating that it is a balance between the drag and magnetic forces that determines the extent of cell retention in the quadrupole system. Additionally, no cell lyses was observed during the experiments, indicating that the clarification technique employing the quadrupole system is gentle enough for the removal of whole, undamaged cells from fermentation broth.

### INCORPORATION BY REFERENCE

All of the patents and publications cited herein are hereby incorporated by reference.

### Equivalents

Those skilled in the art will recognize, or be able to ascertain using no more than routine experimentation, many equivalents to the specific embodiments of the invention described herein. Such equivalents are intended to be encompassed by the following claims.

1. A counter-current device, comprising a plurality of pairs of magnets selected from the group consisting of permanent magnets and electromagnets; a chain rotated by a motor; a syringe pump; tubing; a feed inlet; and a clarified feed outflow.

2. The counter-current device of claim 1, wherein said device is substantially as depicted in FIGS. 7-10.

3. A quadrupole device, comprising four magnets selected from the group consisting of permanent magnets and electromagnets; and a cylindrical column.

4. The quadrupole device of claim 3, wherein said device is substantially as depicted in FIGS. 12-18.

5. A method of separating non-magnetic particles from a magnetic fluid mixture, comprising the steps of combining a magnetic fluid with a sample comprising non-magnetic particles and non-magnetic fluid to form a mixture; subjecting said mixture to a degrading magnetic field; and isolating a portion of the non-magnetic particles from said mixture.

6. The method of claim 5, wherein said magnetic fluid comprises a suspension of magnetic nanoparticles selected from the group consisting of iron, cobalt, nickel, chromium, titanium, manganese, aluminum, copper, samarium, neodymium, iron-nickel, iron-cobalt, iron-copper, iron-copper-aluminum, magnetite, cobalt ferrite, or nickel ferrite in a carrier fluid.

7. The method of claim 5, wherein said magnetic fluid comprises magnetite nanoparticles.

8. The method of claim 5, wherein said magnetic fluid comprises magnetite nanoparticles that are a single crystal of magnetite coated with a graft copolymer layer.

9. The method of claim 5, wherein said magnetic fluid comprises a carrier fluid selected from the group consisting of protic solvents such as water and alcohols or aprotic solvents such as siloxanes, halogenated solvents, or aliphatic or aromatic solvents.

10. The method of claim 5, wherein said magnetic fluid comprises a carrier fluid that is water.

11. The method of claim 5, wherein said magnetic fluid comprises magnetite nanoparticles coated with a graft copolymer layer in water.

12. The method of claim 5, wherein said sample comprises non-magnetic particles and non-magnetic fluid.

13. The method of claim 5, wherein said sample comprises non-magnetic particles that are cells.

14. The method of claim 5, wherein said sample comprises non-magnetic particles that are prokaryotic cells.

15. The method of claim 5, wherein said sample comprises non-magnetic particles that are bacterial cells.

16. The method of claim 5, wherein said sample comprises non-magnetic particles that are E. coli cells.

17. The method of claim 5, wherein said sample comprises non-magnetic particles that are eukaryotic cells.

18. The method of claim 5, wherein said sample comprises non-magnetic particles that are eukaryotic cells selected from a group consisting of cultured mammalian cells, yeast cells or other fungal cells, insect cells, or plant cells.

19. The method of claim 5, wherein said sample comprises non-magnetic particles that are cultured mammalian cells.

20. The method of claim 5, wherein said sample comprises non-magnetic particles that are yeast cells.

21. The method of claim 5, wherein said sample comprises non-magnetic fluid selected from the group consisting of fermentation broth, growth media, or culture media.

22. The method of claim 5, wherein said sample comprises non-magnetic fluid that is fermentation broth.

23. The method of claim 5, wherein said sample comprises a viral particle.

24. The method of claim 5, wherein said sample comprises a subcellular organelle.

25. The method of claim 5, wherein said degrading magnetic field is provided by a counter-current device of claim 1 or 2.

26. The method of claim 5, wherein said degrading magnetic field is provided by a quadrupole device of claim 3 or 4.
